(19) United States
(12) Patent Application Publication
(10) Pub. No.: US 2006/0122236 A1
(43) Pub. Date: Jun. 8, 2006

(54) Substituted biaryl-carboxylate derivatives

(76) Inventors: Michael R. Wood, Harleysville, PA (US); Mark G. Bock, Hatfield, PA (US); Kathy M. Books, Quakertown, PA (US); Roger M. Freidinger, Lansdale, PA (US); June J. Kim, Collegeville, PA (US)

Correspondence Address: MERCK AND CO., INC, P O BOX 2000, RAHWAY, NJ, 07065-0907, US

(21) Appl. No.: 11/291,560

(22) Filed: Dec. 1, 2005

Related U.S. Application Data
(60) Provisional application No. 60/633,577, filed on Dec. 6, 2004.
(60) Provisional application No. 60/636,497, filed on Dec. 16, 2004.

(51) Int. Cl. A61K 31/44; A61K 31/195; A61K 31/24; C07D 213/72

(52) U.S. Cl. 514/352; 514/567; 546/312; 560/19; 514/538

(57) ABSTRACT

Substituted biaryl carboxylate derivatives are bradykinin B1 antagonists or inverse agonists useful in the treatment or prevention of symptoms such as pain and inflammation associated with the bradykinin B1 pathway.

### BACKGROUND OF THE INVENTION

This invention is directed to substituted biaryl carboxylate compounds. In particular, this invention is directed to substituted biaryl carboxylate compounds that are bradykinin antagonists or inverse agonists.

Bradykinin (“BK”) is a kinin which plays an important role in the pathophysiological processes accompanying acute and chronic pain and inflammation. Bradykinin (BK), like other kinins, is an autacoid peptide produced by the catalytic action of kallikrein enzymes on plasma and tissue precursors termed kininogens. The biological actions of BK are mediated by at least two major G-protein-coupled BK receptors termed B1 and B2. It is generally believed that B2 receptors, but not B1 receptors, are expressed in normal tissues and that inflammation, tissue damage or bacterial infection can rapidly induce B1 receptor expression. This makes the B1 receptor a particularly attractive drug target. The putative role of kinins, and specifically BK, in the management of pain and inflammation has provided the impetus for developing potent and selective BK antagonists. In recent years, this effort has been heightened with the expectation that useful therapeutic agents with analgesic and anti-inflammatory properties would provide relief from maladies mediated through a BK receptor pathway (see e.g., M. G. Bock and J. Longmore, Current Opinion in Chem. Biol., 4:401-406(2000)). Accordingly, there is a need for novel compounds that are effective in blocking or reversing activation of bradykinin receptors. Such compounds would be useful in the management of pain and inflammation, as well as in the treatment or prevention of diseases and disorders mediated by bradykinin; further, such compounds are also useful as research tools (in vivo and in vitro).

Canadian Published Application No. 2,050,769 discloses compounds of the formula: 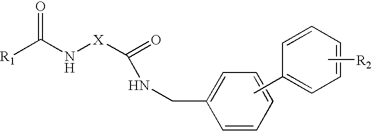
which are intermediates in the preparation of angiotensin II antagonists.

### SUMMARY OF THE INVENTION

The present invention provides substituted biaryl carboxylate compounds which are bradykinin B1 antagonists or inverse agonists, pharmaceutical compositions containing such compounds, and methods of using them as therapeutic agents.

### DETAILED DESCRIPTION OF THE INVENTION

The present invention provides compounds of Formula I and pharmaceutically acceptable salts thereof: 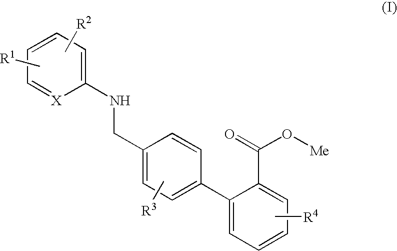
wherein
X is CH or N;
R¹ is selected from
(1) NR^{b}SO₂R^{d},
(2) NR^{b}C(O)-phenyl optionally substituted with 1 to 3 groups independently selected from OR^{a}, nitro, halogen, C₁₋₃ haloalkyl, C₁₋₄ alkyl, (CH₂)ₖNR^{b}R^{c}, cyano, SO₂NR^{b}R^{c}, CO₂R^{a}, C(O)NR^{b}R^{c}, 4,5-dihydro-1H-imidazolyl, and C₃₋₆ cycloalkyl,
(3) nitro,
(4) cyano,
(5) 4,5-dihydro-1H-imidazolyl,
(6) S(O)ᵥR^{d},
(7) SO₂NR^{b}R^{c},
(8) C(O)R^{d},
(9) CO₂R^{a}, and
(10) C(O)NR^{b}R^{c},
   R² is selected from hydrogen, halogen, cyano, nitro, OR^{a}, R^{a}, and C₁₋₄ alkyl,
   R³ and R⁴ are independently selected from hydrogen, halogen, and C₁₋₄ alkyl optionally
   substituted with 1 to 5 halogen atoms;
   R^{a} is selected from (1) hydrogen, (2) C₁₋₄ alkyl optionally substituted with 1 to 5 groups independently selected from halogen, NR^{e}R^{f} and heterocycle, (3) (CH₂)ₖ-phenyl optionally substituted with 1 to 3 groups independently selected from halogen, cyano, nitro, OH, C₁₋₄ alkyloxy, C₃₋₆ cycloalkyl and C₁₋₄ alkyl optionally substituted with 1 to 5 halogen atoms, (4) C₃₋₆ cycloalkyl, and (5) heteroaryl;
   R^{b} and R^{c} are independently selected from
(1) hydrogen,
(2) C₁₋₄ alkyl optionally substituted with 1 to 5 groups independently selected from halogen, heterocycle, cyano, C₃₋₆ cycloalkyl, NR^{e}R^{f}, C₁₋₄ alkyloxy, CO₂R^{a}, OR^{a}, and SO₂R^{d},
(3) (CH₂)ₖ-phenyl, wherein the phenyl is optionally substituted with 1 to 3 groups selected from halogen, cyano, nitro, NR^{e}R^{f}, OR^{a}, CO₂R^{a}, C₁₋₄ alkyloxy, SO₂NR^{e}R^{f}, C₃₋₆ cycloalkyl and C₁₋₄ alkyl optionally substituted with 1 to 5 halogen atoms,
(4) (CH₂)ₖ-heteroaryl,
(5) (CH₂)ₖ—C₃₋₆ cycloalkyl,
(6) (CH₂)ₖ-heterocycle wherein the heterocycle is optionally substituted with one or two groups independently selected from C₁₋₄alkyl, oxo, benzyl, heterocycle, and OR^{a}, and wherein the heterocycle is a 4-, 5-, or 6-membered ring containing one or more heteroatoms selected from NR^{e}, O, and S, wherein the S is optionally oxidized to the sulfone or sulfoxide; or
   R^{b} and R^{c} together with the nitrogen atom to which they are attached form a 4-, 5-, or 6-membered ring optionally containing an additional heteroatom selected from NR^{e}, O, and S, wherein the S is optionally oxidized to the sulfone or sulfoxide, and wherein said 4-, 5- or 6-membered ring is (a) optionally fused to benzene or a 5- or 6-membered heteraromatic ring optionally substituted with CF₃, or (b) optionally spirofused to a heterocycle containing N-R^{e}, or (c) optionally substituted with one to two groups selected from heteroaryl, CO₂R^{a}, heterycycle, and OR^{a}; or
   R^{b} and R^{c} together with the nitrogen atom to which they are attached form a cyclic imide,
   R^{d} is selected from
(1) C₁₋₄ alkyl optionally substituted with 1 to 5 halogen atoms,
(2) C₁₋₄ alkyloxy, optionally substituted with phenyl, wherein the phenyl is optionally substituted with 1 to 3 heterocycle groups,
(3) phenyl optionally substituted with 1 to 3 groups selected from halogen, cyano, nitro, OR^{a}, CO₂R^{a}, C₁₋₄ alkyloxy, C₃₋₆ cycloalkyl and C₁₋₄ alkyl optionally substituted with 1 to 5 halogen atoms,
(4) pyridyl,
(5) heterocycle optionally substituted with one or more heterocycle groups, and
(6) pyridyl N-oxide;
   R^{e} and R^{f} are independently selected from hydrogen, C₁₋₄ alkyl, heterocycle, CO₂R^{a}, COR^{a}, phenyl and pyridyl, or
   R^{e} and R^{f} together with the nitrogen atom to which they are attached form a 4-, 5-, or 6-membered ring optionally containing an additional heteroatom selected from N, O, and S, wherein the S is optionally oxidized to the sulfone or sulfoxide, and optionally substituted with C₁₋₄alkyl or oxo;
   k is 0, 1, 2,3, or 4; and
   v is 0, 1, or 2.

In a first embodiment of Formula I are compounds wherein R² is hydrogen.

In a second embodiment of Formula I are compounds wherein X is CH.

In a third embodiment of Formula I are compounds wherein X is N.

In a fourth embodiment of Formula I are compounds wherein each of R³ and R⁴ is halogen.

In a further embodiment of Formula I are compounds represented by Formula I(1): 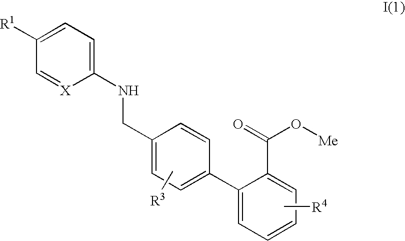
wherein X, R¹, R³, R⁴ and all other variables are defined herein.

In a first subset of Formula I(1) are compounds wherein R¹ is C(O)NR^{b}R^{c}. In a further subset thereof, R^{b} is hydrogen or C₁₋₄ alkyl and R^{c} is selected from (1) (CH₂)ₖ-optionally substituted phenyl, (2) optionally substituted C₁₋₄ alkyl, and (3) (CH₂)ₖ-heterocycle. In another subset thereof, R^{b} is hydrogen. In yet another subset thereof, R^{b} is hydrogen, R^{c} is (CH₂)ₖ-optionally substituted phenyl, and k is 0, 1, or 2. In an additional subset thereof, R^{b} is hydrogen and R^{c} is optionally substituted C₁₋₄ alkyl. In a still further subset thereof, R^{b} is hydrogen and R^{c} is (CH₂)ₖ-heterocycle.

In a second subset of Formula I(1) are compounds wherein R¹ is SO₂NR^{b}R^{c}. In a further subset thereof are compounds wherein R^{b} is hydrogen or C₁₋₄ alkyl and R^{c} is (CH₂)ₖ-optionally substituted phenyl. In an additional subset thereof, R^{b} is hydrogen, R^{c} is (CH₂)ₖ-optionally substituted phenyl, and k is 0.

In a third subset of Formula I(1) are compounds wherein R¹ is S(O)ᵥR^{d}. In a further subset therof are compounds wherein v is 1 or 2, and R^{d} is selected from (1) C₁₋₄ alkyl optionally substituted with 1 to 5 halogen atoms; (2) phenyl optionally substituted with 1 to 3 groups selected from halogen, cyano, nitro, OR^{a}, CO₂R^{a}, C₁₋₄ alkyloxy, C₃₋₆ cycloalkyl and C₁₋₄ alkyl optionally substituted with 1 to 5 halogen atoms; (3) pyridyl; and (4) pyridyl N-oxide. In a further subset thereof, R^{d} is phenyl substituted with CO₂R^{a}. In a yet further subset thereof, R^{d} is phenyl substituted with 1 to 3 halogen groups. In a still further subset thereof. R^{d} is C₁₋₄ alkyl.

In an additional subset of Formula I(1) are compounds wherein each of R³ and R⁴ is halogen.

In another embodiment of Formula I are compounds represented by Formula I(2): 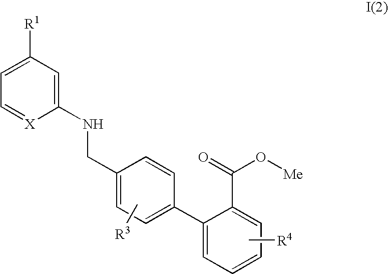
wherein X, R¹, R³, R⁴ and all other variables are defined herein.

In a subset of Formula I(2) are compounds wherein R¹ is C(O)NR^{b}R^{c}. In a further subset thereof are compounds wherein X is CH, R^{b} is hydrogen or C₁₋₄ alkyl and R^{c} is selected from (1) (CH₂)ₖ-optionally substituted phenyl, (2) optionally substituted C₁₋₄ alkyl, and (3) (CH₂)ₖ-heterocycle. In a further subset thereof, R^{b} is hydrogen. In yet another subset thereof, R^{b} is hydrogen, R^{c} is (CH₂)ₖ-heterocycle, and k is 1 or 2.

In a second subset of Formula I(2) are compounds wherein R¹ is SO₂NR^{b}R^{c}. In a further subset thereof are compounds wherein X is N.

In a third subset of Formula I(2) are compounds wherein R¹ is S(O)ᵥR^{d}. In a further subset thereof are compounds wherein X is N.

In an additional subset of Formula I(2) are compounds wherein each of R³ and R⁴ is halogen.

Unless otherwise stated, the following terms have the meanings indicated below:

“Alkyl” as well as other groups having the prefix “alk” such as, for example, alkoxy, alkanoyl, alkenyl, alkynyl and the like, means carbon chains which may be linear or branched or combinations thereof. Examples of alkyl groups include methyl, ethyl, propyl, isopropyl, butyl, sec- and tert-butyl, pentyl, hexyl, heptyl and the like.

“Alkynyl” means a linear or branched carbon chain containing at least one C≡C bond. Examples of alkynyl include propargyl, 2-butynyl, 3-butynyl, 1-methyl-2-propynyl, and the like.

“Aryl” means an aromatic carbocycle having from 6 to 10 carbon atoms, optionally fused to a C₄-C₆ non-aromatic ring optionally containing 1-3 heteroatoms selected from N, 0 and S. Examples of aryl groups include phenyl and naphthyl.

“Halogen” means fluorine, chlorine, bromine and iodine.

“Heteroaryl” means means a monocyclic or bicyclic ring of up to 10 atoms wherein at least one ring is aromatic and contains from 1 to 4 heteroatoms selected from the group consisting of O, N and S. Heteroaryl groups within the scope of this definition include, but are not limited to, pyrrolyl, imidazolyl, pyrazolyl, pyridyl, pyrimidinyl, pyrazinyl, pyridazinyl, furanyl, thienyl, oxazolyl, isoxazolyl, oxadiazolyl, thiazolyl, isothiazolyl, thiadiazolyl, triazolyl, tetrazolyl, indolyl, isoindolyl, benzimidazolyl, benzofuranyl, benzothienyl, benzofurazanyl, benzopyrazolyl, benzotriazolyl, benzoxazolyl, benzothiazolyl, benzisoxazolyl, benzisothiazolyl, quinolinyl, isoquinolinyl, cinnolinyl, quinazolinyl, quinoxalinyl, indolinyl, indolazinyl, indazolyl, isobenzofuranyl, naphthyridinyl, tetrazolopyridyl, dihydrobenzoimidazolyl, dihydrobenzofuranyl, dihydrobenzothiophenyl, dihydrobenzoxazolyl, dihydroindolyl, dihydroquinolinyl, tetrahydroquinolinyl. In cases where the heteroaryl substituent is bicyclic and one ring is non-aromatic (e.g, cycloalkyl, cycloalkenyl or heterocyclyl), it is understood that attachment is via the heteroaromatic ring; if both rings are aromatic and one contains no heteroatom, the attachment can be via either ring. If the heteroaryl contains nitrogen atoms, it is understood that the corresponding N-oxides thereof are also encompassed by this definition.

“Heterocycle” means mono- or bicyclic compounds that are saturated or partly unsaturated, as well as benzo- or heteroaromatic ring fused saturated heterocycles or partly unsaturated heterocycles, and containing from 1 to 4 heteroatoms independently selected from oxygen, sulfur and nitrogen. Examples of saturated heterocycles include morpholine, thiomorpholine, piperidine, piperazine, tetrahydropyran, tetrahydrofuran, dioxane, tetrahydrothiophene, oxazolidine, pyrrolidine; examples of partly unsaturated heterocycles include dihydropyran, dihydropyridazine, dihydrofuran, dihydrooxazole, dihydropyrazole, dihydropyridine, dihydropyridazine and the like. Examples of benzo- or heteroaromatic ring fused heterocycle include 2,3-dihydrobenzofuranyl, naphthyridine, benzopyranyl, tetrahydro-quinoline, tetrahydroisoquinoline, benzomorpholinyl, 1,4-benzodioxanyl, 2,3-dihydrofuro(2,3-b)pyridyl and the like.

“Optionally substituted” is intended to include both substituted and unsubstituted. Thus, for example, optionally substituted aryl could represent a pentafluorophenyl or a phenyl ring.

Optical Isomers—Diastereomers—Geometric Isomers—Tautomers

Compounds described herein may contain an asymmetric center and may thus exist as enantiomers. Where the compounds according to the invention possess two or more asymmetric centers, they may additionally exist as diastereomers. The present invention includes all such possible stereoisomers as substantially pure resolved enantiomers, racemic mixtures thereof, as well as mixtures of diastereomers. The above Formula I is shown without a definitive stereochemistry at certain positions. The present invention includes all stereoisomers of Formula I and pharmaceutically acceptable salts thereof. Diastereoisomeric pairs of enantiomers may be separated by, for example, fractional crystallization from a suitable solvent, and the pair of enantiomers thus obtained may be separated into individual stereoisomers by conventional means, for example by the use of an optically active acid or base as a resolving agent or on a chiral HPLC column. Further, any enantiomer or diastereomer of a compound of the general Formula I may be obtained by stereospecific synthesis using optically pure starting materials or reagents of known configuration.

Some of the compounds described herein contain olefinic double bonds, and unless specified otherwise, are meant to include both E and Z geometric isomers.

Some of the compounds described herein may exist with different points of attachment of hydrogen, referred to as tautomers. Such an example may be a ketone and its enol form known as keto-enol tautomers. The individual tautomers as well as mixture thereof are encompassed with compounds of Formula I.

Salts

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Preferred are the ammonium, calcium, magnesium, potassium and sodium salts. Salts prepared from pharmaceutically acceptable organic non-toxic bases include salts of primary, secondary, and tertiary amines derived from both naturally occurring and synthetic sources. Pharmaceutically acceptable organic non-toxic bases from which salts can be formed include, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, dicyclohexylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from pharmaceutically acceptable non-toxic inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluene-sulfonic acid and the like. Preferred are citric, hydrobromic, hydrochloric, maleic, phosphoric, sulfuric, and tartaric acids.

Prodrugs

The present invention includes within its scope prodrugs of the compounds of this invention. In general, such prodrugs will be functional derivatives of the compounds of this invention which are readily convertible in vivo into the required compound. Thus, in the methods of treatment of the present invention, the term “administering” shall encompass the treatment of the various conditions described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs,” ed. H. Bundgaard, Elsevier, 1985. Metabolites of these compounds include active species produced upon introduction of compounds of this invention into the biological milieu.

Pharmaceutical Compositions

Another aspect of the present invention provides pharmaceutical compositions which comprises a compound of Formula I and a pharmaceutically acceptable carrier. The term “composition”, as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of Formula I, additional active ingredient(s), and pharmaceutically acceptable excipients.

The pharmaceutical compositions of the present invention comprise a compound represented by Formula I (or pharmaceutically acceptable salts thereof) as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds represented by Formula I, or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compound represented by Formula I, or pharmaceutically acceptable salts thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound or a pharmaceutically acceptable salt of Formula I. The compounds of Formula I, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.1 mg to about 500 mg of the active ingredient and each cachet or capsule preferably containing from about 0.1 mg to about 500 mg of the active ingredient.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound represented by Formula I of this invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in moulds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound described by Formula I, or pharmaceutically acceptable salts thereof, may also be prepared in powder or liquid concentrate form.

The following are examples of representative pharmaceutical dosage forms for the compounds of Formula I:

| Injectable Suspension (I.M.) | mg/mL |
|---|---|
| Compound of Formula I | 10 |
| Methylcellulose | 5.0 |
| Tween 80 | 0.5 |
| Benzyl alcohol | 9.0 |
| Benzalkonium chloride | 1.0 |

| Water for injection to a total volume of 1 mL | |
|---|---|
| Tablet | mg/tablet |
| Compound of Formula I | 25 |
| Microcrystalline Cellulose | 415 |
| Povidone | 14.0 |
| Pregelatinized Starch | 43.5 |
| Magnesium Stearate | 2.5 |
| | 500 |

| Capsule | mg/capsule |
|---|---|
| Compound of Formula I | 25 |
| Lactose Powder | 573.5 |
| Magnesium Stearate | 1.5 |
| | 600 |

Utilities

Compounds of this invention are antagonists or inverse agonists of bradykinin receptor, in particular the bradykinin B1 receptor, and as such are useful in the treatment and prevention of diseases and conditions mediated through the bradykinin receptor pathway such as pain and inflammation. The compounds would be effective in the treatment or prevention of pain including, for example, visceral pain (such as pancreatitis, interstitial cystitis, renal colic, prostatitis, chronic pelvic pain), neuropathic pain (such as postherpetic neuralgia, acute zoster pain, nerve injury, the “dynias”, e.g., vulvodynia, phantom limb pain, root avulsions, radiculopathy, painful traumatic mononeuropathy, painful entrapment neuropathy, carpal tunnel syndrome, ulnar neuropathy, tarsal tunnel syndrome, painful diabetic neuropathy, painful polyneuropathy, trigeminal neuralgia), central pain syndromes (potentially caused by virtually any lesion at any level of the nervous system including but not limited to stroke, multiple sclerosis, spinal cord injury), and postsurgical pain syndromes (eg, postmastectomy syndrome, postthoracotomy syndrome, stump pain)), bone and joint pain (osteoarthritis), spine pain (e.g., acute and chronic low back pain, neck pain, spinal stenosis), shoulder pain, repetitive motion pain, dental pain, sore throat, cancer pain, myofascial pain (muscular injury, fibromyalgia), postoperative, perioperative pain and preemptive analgesia (including but not limited to general surgery, orthopedic, and gynecological), chronic pain, dysmenorrhea (primary and secodnary), as well as pain associated with angina, and inflammatory pain of varied origins (e.g. osteoarthritis, rheumatoid arthritis, rheumatic disease, teno-synovitis and gout, ankylosing spondylitis, bursitis).

Further, the compounds of this invention can also be used to treat hyperreactive airways and to treat inflammatory events associated with airways disease e.g. asthma including allergic asthma (atopic or non-atopic) as well as exercise-induced bronchoconstriction, occupational asthma, viral- or bacterial exacerbation of asthma, other non-allergic asthmas and “wheezy-infant syndrome”. Compounds of the present invention may also be used to treat chronic obstructive pulmonary disease including emphysema, adult respiratory distress syndrome, bronchitis, pneumonia, allergic rhinitis (seasonal and perennial), and vasomotor rhinitis. They may also be effective against pneumoconiosis, including aluminosis, anthracosis, asbestosis, chalicosis, ptilosis, siderosis, silicosis, tabacosis and byssinosis.

Compounds of the present invention may also be used for the treatment of inflammatory bowel disease including Crohn's disease and ulcerative colitis, irritable bowel syndrome, pancreatitis, nephritis, cystitis (interstitial cystitis), uveitis, inflammatory skin disorders such as psoriasis and eczema, rheumatoid arthritis and edema resulting from trauma associated with burns, sprains or fracture, cerebral edema and angioedema. They may be used to treat diabetic vasculopathy, diabetic neuropathy, diabetic retinopathy, post capillary resistance or diabetic symptoms associated with insulitis (e.g. hyperglycemia, diuresis, proteinuria and increased nitrite and kallikrein urinary excretion). They may be used as smooth muscle relaxants for the treatment of spasm of the gastrointestinal tract or uterus. Additionally, they may be effective against liver disease, multiple sclerosis, cardiovascular disease, e.g. atherosclerosis, congestive heart failure, myocardial infarct; neurodegenerative diseases, eg. Parkinson's and Alzheimers disease, epilepsy, septic shock e.g. as anti-hypovolemic and/or anti-hypotensive agents, headache including cluster headache, migraine including prophylactic and acute use, stroke, closed head trauma, cancer, sepsis, gingivitis, osteoporosis, benign prostatic hyperplasia and hyperactive bladder. Animal models of these diseases and conditions are generally well known in the art, and may be suitable for evaluating compounds of the present invention for their potential utilities. Finally, compounds of the present invention are also useful as research tools (in vivo and in vitro).

The compounds of this invention are useful in the treatment of pain and inflammation by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

The compounds would be effective in the treatment or prevention of pain including, for example, bone and joint pain (osteoarthritis), repetitive motion pain, dental pain, cancer pain, myofascial pain (muscular injury, fibromyalgia), perioperative pain (general surgery, oral surgery, gynecological), neuropathic pain (post-herpetic neuralgia), and chronic pain by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

In particular, inflammatory pain such as, for example, inflammatory airways disease (chronic obstructive pulmonary disease) would be effectively treated by the compounds of this invention by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

Further, the compounds of this invention can additionally be used to treat asthma, inflammatory bowel disease, rhinitis, pancreatitis, cystitis (interstitial cystitis), uveitis, inflammatory skin disorders, rheumatoid arthritis and edema resulting from trauma associated with burns, sprains or fracture by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

They may be used subsequent to surgical intervention (e.g. as post-operative analgesics) and to treat inflammatory pain of varied origins (e.g. osteoarthritis, rheumatoid arthritis, rheumatic disease, teno-synovitis and gout) as well as for the treatment of pain associated with angina, menstruation or cancer by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

They may be used to treat diabetic vasculopathy, post capillary resistance or diabetic symptoms associated with insulitis (e.g. hyperglycemia, diuresis, proteinuria and increased nitrite and kallikrein urinary excretion) by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

They may be used to treat inflammatory skin disorders such as psoriasis and eczema by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

They may be used as smooth muscle relaxants for the treatment of spasm of the gastrointestinal tract or uterus or in the therapy of Crohn's disease, ulcerative colitis or pancreatitis by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

Such compounds may be used therapeutically to treat hyperreactive airways and to treat inflammatory events associated with airways disease e.g. asthma, and to control, restrict or reverse airways hyperreactivity in asthma by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

They may be used to treat intrinsic and extrinsic asthma including allergic asthma (atopic or non-atopic) as well as exercise-induced bronchoconstriction, occupational asthma, viral or bacterial exacerbated asthma, other non-allergic asthmas and “wheezy-infant syndrome” by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

They may also be effective against pneumoconiosis, including aluminosis, anthracosis, asbestosis, chalicosis, ptilosis, siderosis, silicosis, tabacosis and byssinosis was well as adult respiratory distress syndrome, chronic obstructive pulmonary or airways disease, bronchitis, allergic rhinitis, and vasomotor rhinitis by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

Additionally, they may be effective against liver disease, multiple sclerosis, atherosclerosis, Alzheimer's disease, septic shock e.g. as anti-hypovolemic and/or anti-hypotensive agents, cerebral edema, headache including cluster headache, migraine including prophylactic and acute use, closed head trauma, irritable bowel syndrome and nephritis by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

Combination Therapy

Compounds of Formula I may be used in combination with other drugs that are used in the treatment/prevention/suppression or amelioration of the diseases or conditions for which compounds of Formula I are useful. Such other drugs may be administered, by a route and in an amount commonly used therefor, contemporaneously or sequentially with a compound of Formula I. When a compound of Formula I is used contemporaneously with one or more other drugs, a pharmaceutical composition containing such other drugs in addition to the compound of Formula I is preferred. Accordingly, the pharmaceutical compositions of the present invention include those that also contain one or more other active ingredients, in addition to a compound of Formula I. Examples of other active ingredients that may be combined with a compound of Formula I, either administered separately or in the same pharmaceutical compositions, include, but are not limited to: (1) morphine and other opiate receptor agonists including propoxyphene (Darvon) and tramadol; (2) non-steroidal antiinflammatory drugs (NSAIDs) including COX-2 inhibitors such as propionic acid derivatives (alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone), and the coxibs (celecoxib, valecoxib, rofecoxib and etoricoxib); (3) corticosteroids such as betamethasone, budesonide, cortisone, dexamethasone, hydrocortisone, methylprednisolone, prednisolone, prednisone and triamcinolone; (4) histamine H1 receptor antagonists such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, cetirizine, desloratadine, fexofenadine and levocetirizine; (5) histamine H2 receptor antagonists such as cimetidine, famotidine and ranitidine; (6) proton pump inhibitors such as omeprazole, pantoprazole and esomeprazole; (7) leukotriene antagonists and 5-lipoxygenase inhibitors such as zafirlukast, montelukast, pranlukast and zileuton; (8) drugs used for angina, myocardial ischemia including nitrates such as nitroglycerin and isosorbide nitrates, beta blockers such as atenolol, metoprolol, propranolol, acebutolol, betaxolol, bisoprolol, carteolol, labetalol, nadolol, oxprenolol, penbutolol, pindolol, sotalol and timolol, and calcium channel blockers such as diltiazam, verapamil, nifedipine, bepridil, felodipine, flunarizine, isradipine, nicardipine and nimodipine; (9) incontinence medications such as antimuscarinics, e.g., tolterodine and oxybutinin); (10) gastrointestinal antispasmodics (such as atropine, scopolamine, dicyclomine, antimuscarinics, as well as diphenoxylate); skeletal muscle relaxants (cyclobenzaprine, carisoprodol, chlorphenesin, chlorzoxazone, metaxalone, methocarbamol, baclofen, dantrolene, diazepam, or orphenadrine); (11) gout medications such as allopurinol, probenicid and colchicine; (12) drugs for rheumatoid arthritis such as methotrexate, auranofin, aurothioglucose and gold sodium thiomalate; (13) drugs for osteoporosis such as alendronate and raloxifene; decongestants such as pseudoephedrine and phenylpropanolamine; (14) local anesthetics; (15) anti-herpes drugs such as acyclovir, valacyclovir and famcyclovir; (16) anti-emetics such as ondansetron and granisetron; (17) migraine drugs such as the triptans (e.g. rizatriptan, sumatriptan), ergotamine, dihydroergotamine, CGRP antagonists, antidepressants (e.g., tricyclic antidepressants, serotonin-selective reuptake inhibitors, beta-adrenergic blockers); (18) VR1 antagonsits; (19) anticonvulsants (e.g., gabapentin, pregabalin, lamotrigine, topiramate, carbamazepine, oxcarbazepine, phenyloin); (20) glutamate antagonists (e.g., ketamine and other NMDA antagonists, NR2B antagonists); (21) acetaminophen; (22) CCR2 antagonists; (23) PDE4 antagonists.

Biological Evaluation

Assessing the Affinity of Selected Compounds to Bind to the Bradykinin B1 or B2 Receptor

Radioligand binding assays are performed using membranes from CHO cells that stably express the human, rabbit, rat, or dog B1 receptors or CHO cells that express the human B2 receptor. For all receptor types, cells are harvested from culture flasks in PBS/1 mM EDTA and centrifuged at 1000×g for 10 minutes. The cell pellets are homogenized with a polytron in ice cold 20 mM HEPES, 1 mM EDTA, pH 7.4 (lysis buffer) and centrifuged at 20,000×g for 20 minutes. The membrane pellets are rehomogenized in lysis buffer, centrifuged again at 20,000×g and the final pellets are resuspended at 5 mg protein/ml in assay buffer (120 mM NaCl, 5 mM KCl, 20 mM HEPES, pH 7.4) supplemented with 1% BSA and frozen at −80° C.

On the day of assay, membranes are centrifuged at 14,000×g for 5 minutes and resuspended to the desired protein concentration in assay buffer containing 100 nM enaliprilat, 140 μg/mL bacitracin and 0.1% BSA. 3H-des-arg10, leu9 kallidin is the radioligand used for the human and rabbit B1 receptors, 3H-des-arg10 kallidin is used for the rat and dog B1 receptors, and 3H-bradykinin is used to label the human B2 receptor.

For all assays, compounds are diluted from DMSO stock solutions with 4 μL added to assay tubes for a final DMSO concentration of 2%. This is followed by the addition of 100 μL radioligand and 100 μL of the membrane suspension. Nonspecific binding for the B1 receptor binding assays is determined using 1 μM des-arg10 kallidin and nonspecific binding for the B2 receptor is determined with 1 μM bradykinin. Tubes are incubated at room temperature (220C) for 60 minutes followed by filtration using a Tomtec 96-well harvesting system. Radioactivity retained by the filter is counted using a Wallac Beta-plate scintillation counter.

The compounds of this invention have affinity for the B1 receptor in the above assay as demonstrated by results of less than 5 μM. It is advantageous that the assay results be less than 1 μM, even more advantageous for the results be less than 0.5 μM. It is further advantageous that compounds of this invention have affinity for the bradykinin B1 receptor over the bradykinin B2 receptor; more advantageously, the affinity for the B1 receptor is at least 10 fold, and even more advantageously over 100 fold, over that for the B2 receptor.

Assay for Bradykinin B1 Antagonists

B1 agonist-induced calcium mobilization was monitored using a Fluorescence Imaging Plate Reader (FLIPR). CHO cells expressing the B1 receptor were plated in 96 or 384 well plates and allowed to incubate in Iscove's modified DMEM overnight. Wells were washed two times with a physiological buffered salt solution and then incubated with 4 uM Fluo-3 for one hour at 37° C. The plates were then washed two times with buffered salt solution and 100 uL of buffer was added to each well. Plates were placed in the FLIPR unit and allowed to equilibrate for two minutes. The test compound was then added in 50 ul volumes followed five minutes later by 50 ul of agonist (des-arg10 kallidin). Relative fluorescence peak heights in the absence and presence of antagonist were used to calculate the degree of inhibition of the B1 receptor agonist response by the test compound. Eight to ten concentrations of test compound were typically evaluated to construct an inhibition curve and determine IC50 values using a four-parameter nonlinear regression curve fitting routine.

Assay for Bradykinin Inverse Agonists

Inverse agonist activity at the human B1 receptor was evaluated using transiently transfected HEK293 cells. One day following transfection cell flasks were labeled overnight with 6uCi/ml [³H]myo-inositol. On the day of assay, the media was removed and the attached cells were gently rinsed with 2×20 ml of phosphate-buffered saline. Assay buffer (HEPES buffered physiological salts, pH 7.4) was added and the cells were detached by tapping of the flask. The cells were centrifuged at 800×g for five minutes and resuspended at 1×10⁶ cells/ml in assay buffer supplemented with 10 mM lithium chloride. After 10 minutes at room temperature, one-half ml aliquots were distributed to tubes containing test compound or vehicle. After an additional 10 minutes the tubes were transferred to a 37° C. water bath for 30 minutes. The incubation was terminated by the addition of a 12% perchloric acid solution and the tubes were placed on ice for 30 minutes. The acid was then neutralized with KOH and the tubes centrifuged to pellet precipitated material. [3H]Inositol monophosphate formed was recovered by standard ion exchange chromatographic techniques and quantitated by liquid scintillation counting. Inverse agonist activity was determined by the degree to which a test compound reduced basal (cells incubated with vehicle) levels of [³H]inositol monophosphate accumulation.

Abbreviations Used

The following abbreviations have the meanings indicated, unless stated otherwise in the specification: BOC (boc)=t-butyloxycarbonyl; DCM=dichloromethane; DMF=dimethylformamide; DMSO=Dimethyl sulfoxide; EDC or EDCI=1-(3-dimethylaminopropyl)₃-ethylcarbodiimide HCl; eq.=equivalent(s); ES (or ESI)-MS=electron spray ionization-mass spectroscopy; Et=ethyl; EtOAc=ethyl acetate; EtOH=ethanol; FAB-MS=fast atom bombardment-mass spectroscopy; HOBt=1-hydroxybenzotriazole hydrate; HPLC=high pressure liquid chromatography; LCMS=Liquid chromatography/mass spectroscopy; LHMDS=lithium bis(trimethylsilyl)amide; Me=methyl; MeOH=Methanol; MHz=megahertz; MsCI=Mesyl chloride; NEt3=Triethylamine; NMR=nuclear magnetic resonance; TFA=trifluoroacetic acid; THF=tetrahydrofuran.

Compounds of Formula I may be prepared according to the following illustrative schemes.

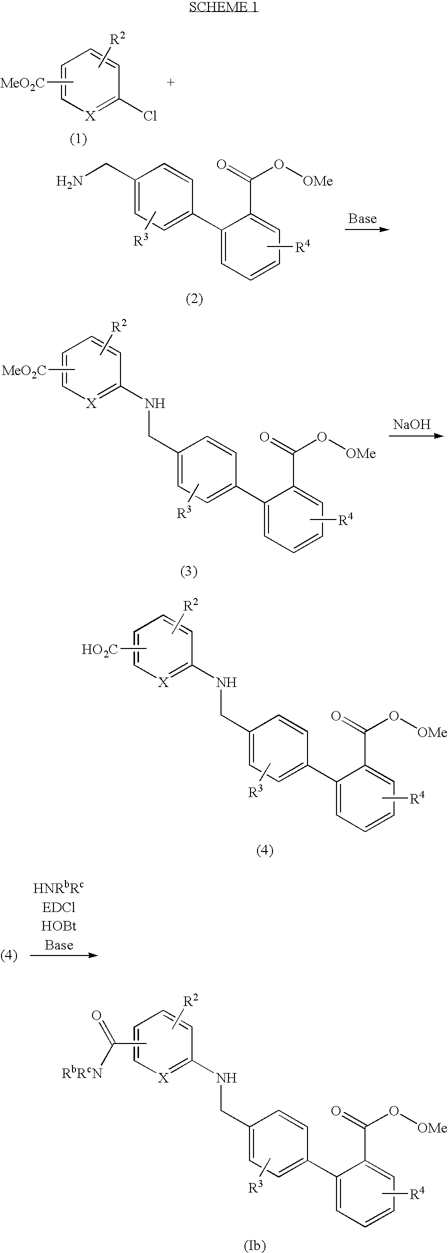

In Scheme 1, the aryl halide of ester (1) is displaced with amine (2), assembled according to procedures described in WO 03/066577, in an appropriate solvent, like methanol, in the presence of an appropriate base, such as triethylamine, at a temperature between 30 and 100° C., to provide (3). Alkaline hydrolysis of (3) in a suitable mixture of water and an organic solvent, like methanol, at a temperature between 0 and 80° C. yields (4). Carboxylic acid (4) is then reacted with an amine (for example, HNR^{b}R^{c}) using standard peptide coupling reagent combinations, such as EDCI/HOBt, in an appropriate solvent, like DCM, in the presence of a tertiary amine base(as needed), such as triethylamine, to provide compound (Ib).

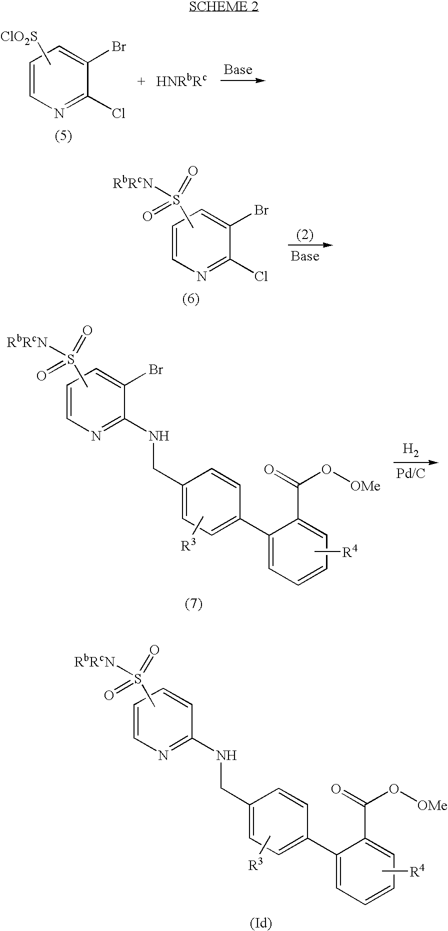

Alternatively, as illustrated in Scheme 2, sulfonyl chloride (5) is reacted with an amine (for example, HNR^{b}R^{c}), in an appropriate solvent, like DCM, in the presence of a tertiary amine base (as needed), such as triethylamine, at a temperature between 0 and 40° C., to provide (6). The aryl chloride of (6) is displaced with amine (2), in an appropriate solvent, like dimethyl sulfoxide, in the presence of an appropriate base, such as triethylamine, at a temperature between 30 and 200° C., to provide (7). The aryl bromide of (7) is reductively cleaved using hydrogen and an appropriate metal catalyst, like Pd/C, in and appropriate solvent, such as methanol, to provide compound (Id).

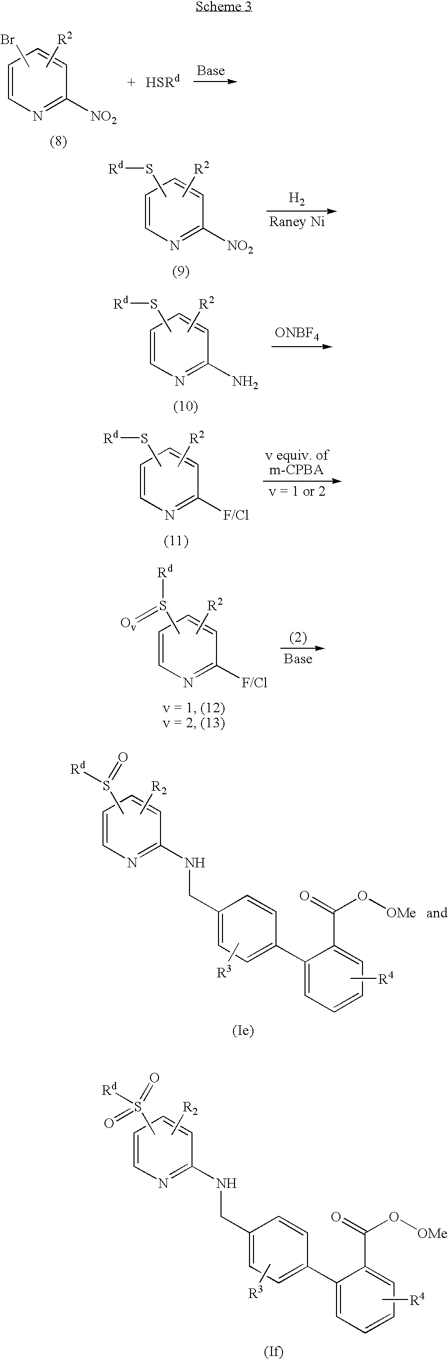

Alternatively, as illustrated in Scheme 3, the aryl halide of (8) is displaced with a thiol, of generic formula HSR^{d}, in an appropriate solvent, like methanol, in the presence of an appropriate base, such as triethylamine, at a temperature between 30 and 50° C., to provide (9). The nitro group of (9) is then reduced using hydrogen and an appropriate metal catalyst, like Raney nickel, in an appropriate solvent, such as methanol at a temperature between 10 and 30° C., to provide (10). The amine (10) is then diazotized with nitrosonium tetrafluoroborate, in an appropriate solvent, like DCM, at a temperature between 0 and 40° C., and following spontaneous decomposition of the diazonium yields (11) as a mixture of aryl chloride and aryl fluoride. The thioether of (11) is then oxidized in an appropriate solvent, like chloroform, at a temperature between −10 and 20° C., using either 1 or 2 equivalents of an appropriate oxidant, such as ni-CPBA, to provide sulfoxide (12) or sulfone (13), respectively. The aryl halides of (12) and (13) are then displaced with amine (2), in an appropriate solvent, like methanol, in the presence of an appropriate base, such as triethylamine, at a temperature between 30 and 100° C., to provide claimed compounds (Ie) and (If), respectively.

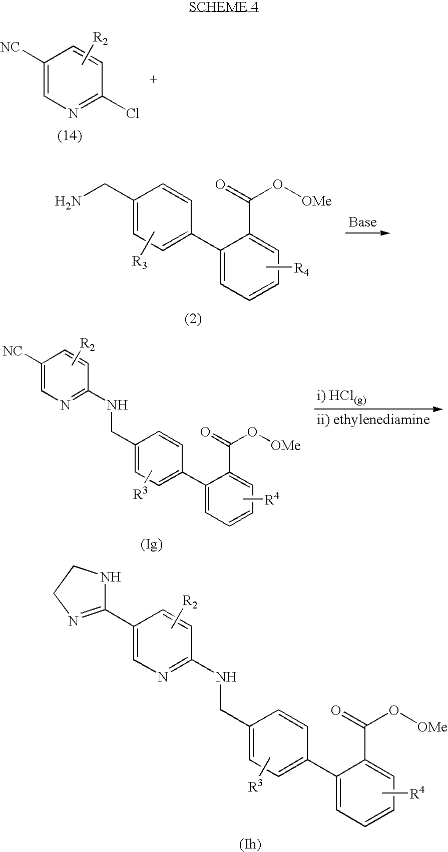

Alternatively, as illustrated in Scheme 4, the aryl halide of nitrile (14) is displaced with amine (2), in an appropriate solvent, like methanol, in the presence of an appropriate base, such as triethylamine, at a temperature between 30 and 100° C., to provide claimed compound (Ig). The cyano group of (Ig) is transformed into an alkyl imidate by the action of a sufficiently strong acid, like HCl, in an appropriate solvent, such as MeOH, and at temperature between 0 and 70° C. This imidate is then reacted with an excess of ethylenediamine, in an appropriate solvent, such as MeOH, at a temperature between 0 and 50° C., to provide (Ih).

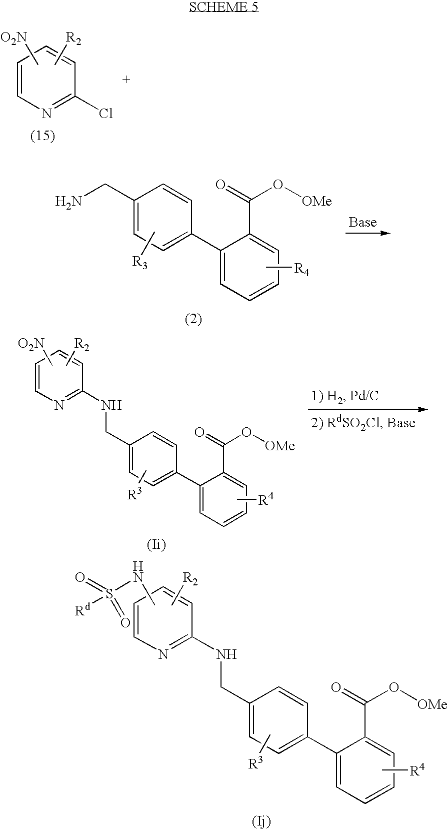

As shown above, the aryl halide of (15) is displaced with amine (2), in an appropriate solvent, like methanol, in the presence of an appropriate base, such as triethylamine, at a temperature between 30 and 80° C., to provide compound (Ii). The nitro group of (Ii) is then reduced using hydrogen and an appropriate metal catalyst, like Pd/C, in an appropriate solvent, such as methanol at a temperature between 10 and 30° C., to provide the coupled amine product, which is then reacted with a sulfonylating reagent (for example, but not limited to, R^{d}SO₂Cl), in an appropriate solvent, like DCM, in the presence of an appropriate base, such as triethylamine, at a temperature between −10 and 30° C., to provide compound (Ij).

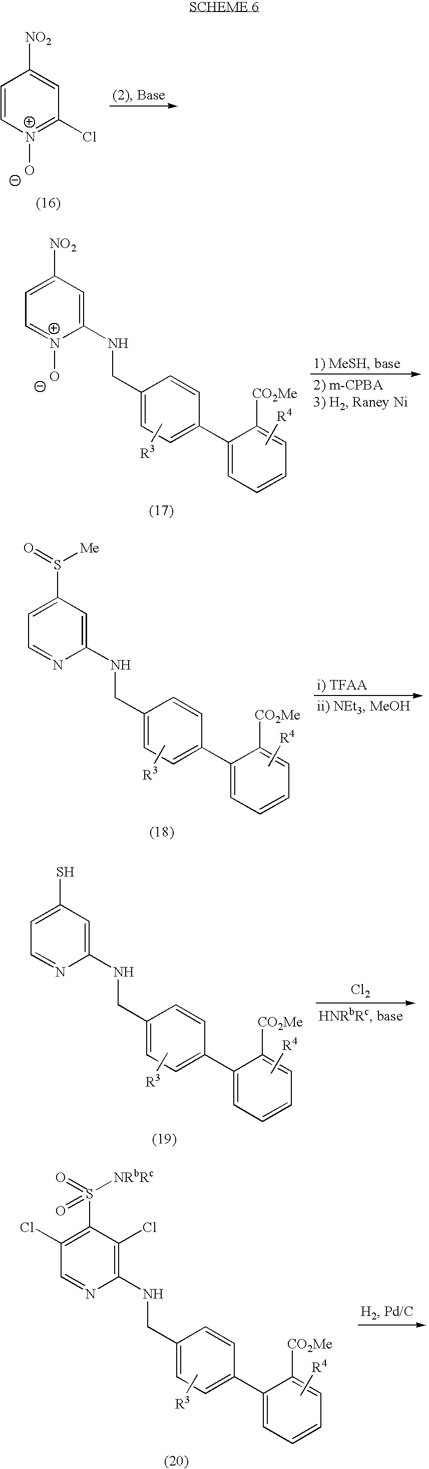

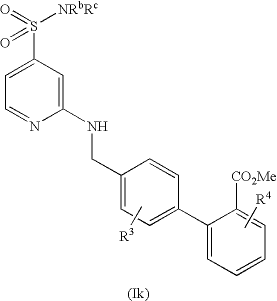

As illustrated in Scheme 6, the aryl chloride (16) is displaced with the amine (2), in the presence of a suitable tertiary amine base, like N-methylmorpholine, in an appropriate solvent, like methanol, at a temperature between 0 and 100° C., to provide intermediate (17). The nitro group of (17) is displaced with methanethiol, in and appropriate solvent, like methanol, at a temperature between 50 and 100° C., in a sealed reaction vessel, in the presence of a suitable tertiary amine base, like N-methyl-morpholine, to provide the corresponding thiomethyl compound. The thiomethyl compound is converted to the sulfoxide (18) by first using an appropriate oxidant, like m-CPBA, in an appropriate solvent, like chloroform, at a temperature between −20 and 20° C., followed by reducing the N-oxide using hydrogen and an appropriate metal catalyst, like Raney nickel, in an appropriate solvent, such as methanol, to provide pyridine (18). The sulfoxide of (18) is then transformed to the thiol (19) using an appropriate Lewis acid, like trifluoroacetic anhydride, at a temperature between 20 and 70° C., followed by cleavage of the initially formed acyl thioacetal using appropriately mild conditions, such as triethylamine in methanol. The thiol of (19) is oxidized to the corresponding sulfonyl chloride using an oxidant, such as chlorine gas, in an appropriate aqueous solvent mixture, such as 1:1H₂O:EtOAc, at a temperature between −10 and 10° C. This sulfonyl chloride is then reacted with an amine (HNR^{b}R^{c}, for example), in an appropriate solvent, like DCM, with the addition of a tertiary amine base, such as triethylamine, at a temperature between 0 and 40° C., to provide (20). The aryl chlorides of (20) can be reductively cleaved using hydrogen and an appropriate metal catalyst, like Pd/C, in and appropriate solvent, such as 2M ammonia in MeOH, to provide compound (Ik).

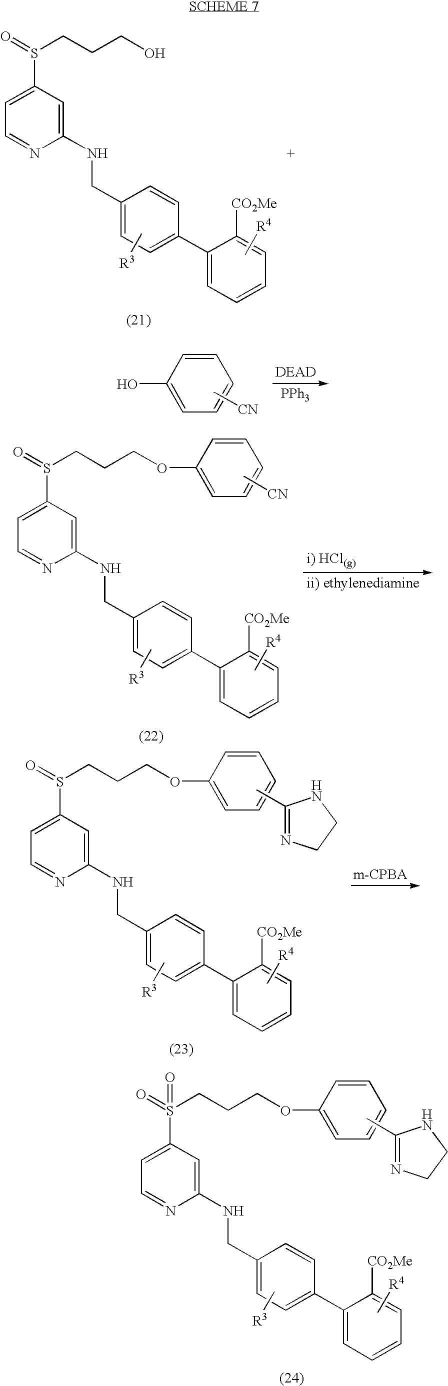

Scheme 7 illustrates additional compounds of the present invention. Compound (21) may be preapred analogously as depicted in Scheme 6; the hydroxy group of (21), is displaced with a cyano-phenol, after activation with a standard Mitsunobu reagent combination, such as triphenylphosphine and DEAD, in an appropriate solvent, such as THF, at a temperature between −10 and 50° C. to provide (22). The cyano group of (22) is transformed into an alkyl imidate by the action of a sufficiently strong acid, like HCl, in an appropriate solvent, such as EtOH, and at temperature between 0 and 70° C. This imidate is then reacted with an excess of ethylenediamine, in an appropriate solvent, such as EtOH, at a temperature between 0 and 50° C., to provide (23). Sulfoxide (23) is further oxidized using an appropriate oxidant, like m-CPBA, in an appropriate solvent, like chloroform, at a temperature between −20 and 20° C. to provide compound (24).

The following examples are provided to illustrate the claimed invention and are not to be construed as limiting the scope thereof in any manner:

### EXAMPLE 1

### Methyl 3,3′-difluoro-4′-{[(5-{[(2-piperidin-1-ylethyl)amino]carbonyl}pyridin-2-yl)amino]methyl}-biphenyl-2-carboxylate

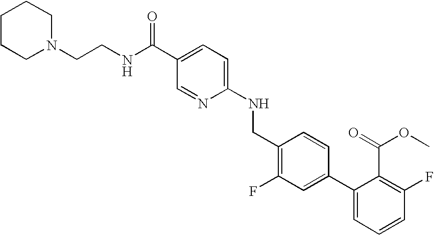

Methyl 6-chloronicotinate (2.60 g, 15.15 mmol), methyl 4′-(aminomethyl)-3,3′-difluorobiphenyl-2-carboxylate (2.10 g, 7.57 mmol, prepared according to procedures described in WO 03/066577) and triethylamine (1.61 g, 15.91 mmol) were dissolved in methanol (50 ml). The reaction vessel was sealed and heated at 80° C. for 7 days. Solvent was removed, and the residue was diluted with ethyl acetate and washed with saturated sodium bicarbonate and brine (×2). The organic layer was dried over sodium sulfate, filtered, and evaporated under reduced pressure to give a residue which was subjected to silica gel chromatography eluted with 10-60% ethyl acetate in hexane to yield methyl 6-({[3,3′-difluoro-2′-(methoxycarbonyl)biphenyl-4-yl]methyl}amino)nicotinate.

To a solution of the above material (1.18 g, 2.86 mmol) in methanol (50 ml) was added 1N NaOH (3.5 ml). After stirring at room temperature for 4 hours, the solution was heated to 50° C. for overnight stirring. Additional 1N NaOH (0.6 ml) was added. After 4 hours, the solution was neutralized by addition of 1N HCl (4.1 ml), and methanol was removed under reduced pressure. The residue was diluted with water, and the precipitates were filtered and dried under vacuum. The solid was purified by silica gel chromatography eluted with 1-10% methanol in methylene chloride to provide 6-({[3,3′-difluoro-2′-(methoxycarbonyl)biphenyl-4-yl]methyl}amino)nicotinic acid.

To a solution of the above material (50 mg, 0.13 mmol), 1-(2-aminoethyl)piperidine (21 mg, 0.16 mmol) and 1-hydroxybenzotriazole hydrate (5.8 mg, 0.04 mmol) in methylene chloride (2 mL) were added 1-(3-dimethylaminopropyl)-3-ethylcarbodiimide hydrochloride (36 mg, 0.19 mmol) and triethylamine (19 mg, 0.19 mmol). After overnight stirring, the mixture was subjected to silica gel chromatography eluted with 1-9% methanol (with 10% NH₄OH) in methylene chloride. Collection of product containing fractions and removal of solvent yielded a residue, which was further purified by reverse phase chromatography eluted with 10-45% acetonitrile in water to provide the title compound as a TFA salt. LRMS (ES, M+H⁺): 509. ¹H NMR (400 MHz, CD₃OD): δ 8.49 (bs, 1H), 8.06 (bd, J=9.2 Hz, 1H), 7.56 (m, 1H), 7.45 (t, J=7.6 Hz, 1H), 7.24 (m, 2H), 7.16 (m, 2H), 6.85 (d, J=9.2 Hz, 1H), 4.72 (s, 2H), 3.73 (t, J=6 Hz, 2H), 3.69 (s, 3H), 3.70-3.66 (m, 2H), 3.32-3.29 (m, 2H), 2.99 (bt, J=12.8 Hz, 2H), 1.98 (m, 2H), 1.80 (m, 3H), 1.55 (m, 1H).

### EXAMPLE 2

### Methyl 3,3′-difluoro-4′-{[(5-{[(2-piperidin-1-ylethyl)amino]sulfonyl}pyridin-2-yl)amino]methyl}-biphenyl-2-carboxylate

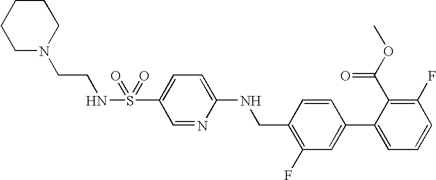

To a solution of 3-bromo-2-chloropyridine-5-sulfonyl chloride (1.0 g, 3.44 mmol) in methylene chloride (4.5 ml) was added 1-(2-aminoethyl)piperidine (0.93 g, 7.22 mmol). After 1 hour of stirring at room temperature, solvent was removed, and the residue was subjected to silica gel chromatography eluted with 2-15% methanol (with 10% NH₄OH) in methylene chloride to provide 5-bromo-6-chloro-N-(2-piperidin-1-ylethyl)pyridine-3-sulfonamide.

The above material (0.55 g, 1.44 mmol) and methyl 4′-(aminomethyl)-3,3′-difluoro-biphenyl-2-carboxylate (0.40 g, 1.44 mmol) were dissolved in DMSO (30 ml) under nitrogen. Triethylamine (0.31 g, 3.03 mmol) was added, and the solution was heated at 150° C. for 2 hours and then cooled to room temperature for continued stirring overnight. The reaction mixture was partitioned between ethyl acetate and saturated sodium bicarbonate, and the organic layer was extracted with additional ethyl acetate. The combined organics were washed with brine, dried over sodium sulfate, filtered, and concentrated. The residue was subjected to silica gel chromatography eluted with 1-8% methanol in methylene chloride to provide methyl 4′-{[(3-bromo-5-{[(2-piperidin-1-ylethyl)amino]-sulfonyl}pyridin-2-yl)amino]methyl}-3,3′-difluorobiphenyl-2-carboxylate.

A stirred solution of the above material (0.13 g, 0.20 mmol) in methanol (2 ml) was purged with nitrogen, and 10% Pd/C catalyst (9 mg) was added. The reaction vessel was again purged with nitrogen and then flushed with hydrogen from a balloon. After 3 days of stirring under hydrogen, nitrogen was bubbled through the solution prior to filtration through a celite pad. The filtrate was concentrated, and the residue was subjected to reverse phase chromatography to provide the title compound as a TFA salt. HRMS (M+H⁺): calc'd 545.2029, found 545.2022. ¹H NMR (400 MHz, CD₃OD): δ 8.45 (bs, 1H), 7.77 (bd, J=8.8 Hz, 1H), 7.55 (m, 1H), 7.43 (t, J=7.6 Hz, 1H), 7.23 (m, 2H), 7.12 (m, 2H), 6.67 (d, J=9.2 Hz, 1H), 4.70 (s, 2H), 3.68 (s, 3H), 3.57 (bd, J=11.6 Hz, 2H), 3.23 (m, 4H), 2.97 (bt, J=12 Hz, 2H), 1.93 (m, 2H), 1.81 (m, 3H), 1.52 (m, 1H).

### EXAMPLE 3

### Methyl 3,3′-difluoro-4′-[({5-[(3-methoxyphenyl)sulfonyl]pyridin-2-yl}amino)methyl]biphenyl-2-carboxylate

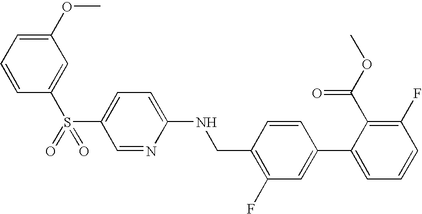

Into a solution of 5-bromo-2-nitropyridine (1.00 g, 4.93 mmol) in methanol (4.0 mL) were added 3-methoxybenzenethiol (0.829 g, 5.91 mmol) and triethylamine (0.598 g, 5.91 mmol). The mixture was stirred at room temperature for 3 days, and solvent was removed under reduced pressure. The residue was partitioned between ethyl acetate and 1N HCl, and the organic layer was washed with brine, dried over sodium sulfate, filtered, and concentrated. The residue was subjected to silica gel chromatography eluted with 100% methylene chloride to provide 5-[(3-methoxyphenyl)thio]-2-nitropyridine.

A solution of the above material (2.50 g, 9.53 mmol) in methanol (35 mL) was purged with nitrogen prior to the addition of Raney Nickel (approximately 2 mL). The mixture was again purged with nitrogen and then with hydrogen from a balloon. After 3 hours of stirring under hydrogen, the mixture was purged with nitrogen prior to filtration through a pad of celite. The filtrate was concentrated to yield 5-[(3-methoxyphenyl)thio]pyridin-2-amine.

Into a solution of the above material (2.20 g, 9.47 mmol) in anhydrous methylene chloride (2.0 mL) at 0° C. was added nitrosonium tetrafluoroborate (1.327 g, 11.36 mmol). The mixture was stirred at 0° C. for 30 minutes and then at room temperature for 2 hours. The reaction was quenched with water, and the mixture was stirred for 5 minutes. The pH of the aqueous layer was adjusted to >7 with saturated sodium bicarbonate, and the product was extracted with two volumes of ethyl acetate. The combined extracts were washed with brine, dried over sodium sulfate, filtered, and concentrated. The residue was subjected to silica gel chromatography eluted with 100% methylene chloride to provide a mixture of 2-fluoro-5-[(3-methoxyphenyl)thio]pyridine and 2-chloro-5-[(3-methoxyphenyl)thio]pyridine.

To a solution of the above material (1.49 g, 6.33 mmol) in chloroform (50 mL) at 0° C. was added mCPBA (2.15 g, 12.5 mmol). Upon completion of the reaction, calcium hydroxide (0.938 g, 12.7 mmol) was added and the resulting mixture was stirred for 30 minutes. The mixture was filtered, and the filtrate was concentrated. The residue was subjected to silica gel chromatography eluted with 0-6% methanol in methylene chloride to afford a mixture of 2-fluoro-5-[(3-methoxyphenyl)sulfonyl]pyridine and 2-chloro-5-[(3-methoxyphenyl)sulfonyl]pyridine.

Into a solution of the above material (0.500 g, 1.87 mmol) and methyl 4′-(aminomethyl)-3,3′-difluorobiphenyl-2-carboxylate (0.571 g, 2.06 mmol) in methanol (11 mL) was added triethylamine (0.398 g, 3.93 mmol). The reaction vessel was sealed and placed in a 50° C. oil bath for 15 hours. The mixture was then heated at 80° C. for additional 24 hours. The mixture was allowed to cool and concentrated under reduced pressure. The residue was diluted with ethyl acetate and washed with saturated sodium bicarbonate and brine. The organic layer was dried over sodium sulfate, filtered, and concentrated to yield a residue, which was subjected to silica gel chromatography eluted with 10-60% ethyl acetate in hexane. The fractions with the desired product were concentrated, and the residue was re-purified by reverse phase chromatography eluted with 60-70% acetonitrile in water to provide the title compound as a TFA salt. HRMS (ES, M+H⁺): calc'd 525.1290, measured 525.1285. ¹H NMR (400 MHz, CD₃OD) δ 8.52 (d, J=2.4 Hz, 1H), 7.79 (dd, J=9.2 and 2.4 Hz, 1H), 7.54 (dt, J=8 and 5.6 Hz, 1H), 7.46 (m, 2H), 7.39 (m, 2H), 7.25-7.14 (m, 3H), 7.09 (m, 2H), 6.61 (d, J=9.2 Hz, 1H), 4.68 (s, 2H), 3.84 (s, 3H), 3.62 (s, 3H).

### EXAMPLE 4

### Methyl 4′-{[(5-cyanopyridin-2-yl)amino]methyl}-3,3′-difluorobiphenyl-2-carboxylate

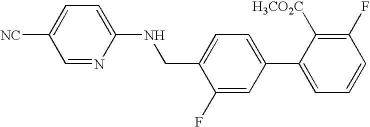

6-Chloronicotinonitrile (0.500 g, 3.61 mmol), methyl 4′-(aminomethyl)-3,3′-difluoro-biphenyl-2-carboxylate (0.500 g, 1.80 mmol) and triethylamine (0.365 g, 3.61 mmol) were dissolved in methanol (5 mL). The reaction vessel was sealed and heated at 80° C. for 5 days. Solvent was removed, and the residue was subjected to silica gel chromatography eluted with 1-5% methanol in methylene chloride. The fractions with the desired product were concentrated, and the residue was re-purified by reverse phase chromatography to provide the title compound as a TFA salt. LRMS (ES, M+H⁺): 380. ¹H NMR (400 MHz, CD₃OD): δ 8.35 (d, J=2.4 Hz, 1H), 7.66 (dd, J=8.8 and 2 Hz, 1H), 7.55 (dt, J=8 and 5.6 Hz, 1H), 7.43 (t, J=7.6 Hz, 1H), 7.23 (m, 2H), 7.12 (bd, J=9.6 Hz, 2H), 6.67 (bd, J=8.8 Hz, 1H), 4.69 (s, 2H), 3.68 (s, 3H).

### EXAMPLE 5

### Methyl 4′-({[5-(4,5-dihydro-1H-imidazol-2-yl)pyridin-2-yl]amino}methyl)-3,3′-difluorobiphenyl-2-carboxylate

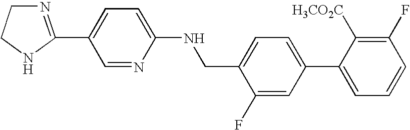

A solution of methyl 4′-{[(5-cyanopyridin-2-yl)amino]methyl}-3,3′-difluorobiphenyl-2-carboxylate (0.193 g, 0.509 mmol, Example 4) in methanol (5 mL) at 0° C. was saturated with anhydrous HCl. The reaction vessel was sealed and warmed to room temperature for overnight stirring. Solvent was removed, and the residue was again dissolved in methanol (5 mL). The solution was cooled to 0° C., and ethylene diamine (0.174 g, 2.91 mmol) was added. The solution was warmed to room temperature for overnight stirring. Solvent was removed, and the residue was purified by reverse phase chromatography to provide the title compound as a TFA salt. LRMS (ES, M+H⁺): 423. ¹H NMR (400 MHz, CD₃OD): δ 8.55 (d, J=2.4 Hz, 1H), 7.77 (dd, J=9.2 Hz, 2.4 Hz, 1H), 7.55 (dt, J=8 and 5.6 Hz, 1H), 7.43 (t, J=8.4 Hz, 1H), 7.23 (m, 2H), 7.12 (m, 2H), 6.69 (d, J=8.8 Hz, 1H), 4.74 (s, 2H), 4.01 (s, 4H), 3.68 (s, 3H).

### EXAMPLE 6

### Methyl 3,3′-difluoro-4′-{[(5-nitropyridin-2-yl)amino]methyl}biphenyl-2-carboxylate

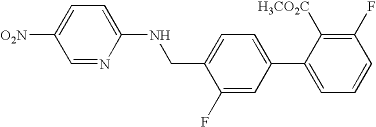

2-Chloro-5-nitropyridine (0.788 g, 4.97 mmol) and methyl 4′-(aminomethyl)-3,3′-difluorobiphenyl-2-carboxylate (1.33 g, 4.80 mmol) were dissolved in methanol (15 mL), and triethylamine (0.838 g, 8.28 mmol) was added. The reaction vessel was sealed and heated at 60° C. for 12 hours. Solvent was removed, and the residue was subjected to silica gel chromatography eluted with 10-70% ethyl acetate in hexane to provide the title compound. HRMS (ES, M+H⁺): calcd 400.1104, measured 400.1078. ¹H NMR (400 MHz, CD₃OD): δ 8.94 (d, J=2.8 Hz, 1H), 8.16 (dd, J=9.2 and 2.8 Hz, 1H), 7.55 (dt, J=8 and 5.6 Hz, 1H), 7.44 (t, J=7.6 Hz, 1H), 7.23 (m, 2H), 7.12 (m, 2H), 6.61 (d, J=9.6 Hz, 1H), 4.75 (s, 2H), 3.68 (s, 3H).

### EXAMPLE 7

### Methyl 3,3′-difluoro-4′-[({5-[(phenylsulfonyl)amino]pyridin-2-yl}amino)methyl]biphenyl-2-carboxylate

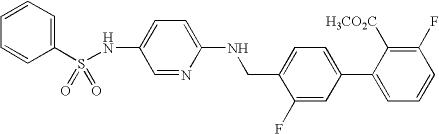

A solution of methyl 3,3′-difluoro-4′-{[(5-nitropyridin-2-yl)amino]methyl}biphenyl-2-carboxylate (0.610 g, 1.53 mmol, Example 6) in methanol (25 mL) was purged with nitrogen prior to the addition of 10% Pd/C catalyst (0.06 g). The mixture was again purged with nitrogen and then with hydrogen from a balloon. After 7 hours of stirring under hydrogen, the mixture was purged with nitrogen and filtered through a celite pad. The filtrate was concentrated to afford methyl 4′-{[(5-aminopyridin-2-yl)amino]methyl}-3,3′-difluorobiphenyl-2-carboxylate.

To a solution of the above material (0.100 g, 0.271 mmol) and triethylamine (0.030 g, 0.298 mmol) in methylene chloride (3 mL) at 0° C. was added benzenesulfonyl chloride (0.050 g, 0.284 mmol). The mixture was stirred at 0° C. for 30 minutes and at room temperature for 1.5 hours. Solvent was removed and the residue was purified by reverse phase chromatography to provide the title compound as a TFA salt. LRMS (ES, M+H⁺): 510. ¹H NMR (400 MHz, CD₃OD): δ 7.76 (m, 2H), 7.67-7.53 (m, 6H), 7.44 (t, J=7.6 Hz, 1H), 7.26 (m, 2H), 7.19 (m, 2H), 6.97 (d, J=9.2 Hz, 1H), 4.62 (s, 2H), 3.69 (s, 3H).

### EXAMPLE 8

### Methyl 4′-({[5-(benzoylamino)pyridin-2-yl]amino}methyl)-3,3′-difluorobiphenyl-2-carboxylate

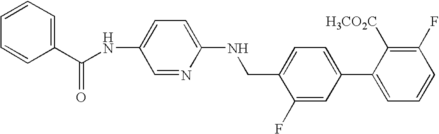

To a solution of methyl 4′-{[(5-aminopyridin-2-yl)amino]methyl}-3,3′-difluorobiphenyl-2-carboxylate (0.075 g, 0.203 mmol, prepared according to Example 7) and triethylamine (0.029 g, 0.284 mmol) in methylene chloride (2 mL) at 0° C. was added benzoyl chloride (0.034 g, 0.244 mmol). After 30 minutes, the mixture was diluted with ethyl acetate and washed with water, saturated sodium bicarbonate, half brine and brine. The organic layer was dried over sodium sulfate, filtered, and concentrated. The residue was subjected to silica gel chromatography eluted with 0-5% methanol in methylene chloride. The fractions with the desired product were concentrated, and the residue was re-purified by reverse phase chromatography to provide the title compound as a TFA salt. LRMS (ES, M+H⁺): 474. ¹H NMR (400 MHz, CD₃OD): δ 8.62 (d, J=2.4 Hz, 1H), 8.09 (dd, J=9.6 and 2.4 Hz, 1H), 7.94 (m, 2H), 7.63-7.48 (m, 5H), 7.27 (d, J=8 Hz, 2H), 7.22 (m, 2H), 7.12 (d, J=9.6 Hz, 1H), 4.71 (s, 2H), 3.71 (s, 3H).

### EXAMPLE 9

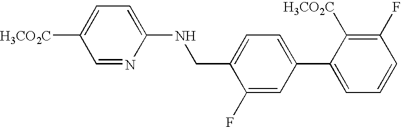

LRMS (ES, M+H⁺): 413

### EXAMPLE 10

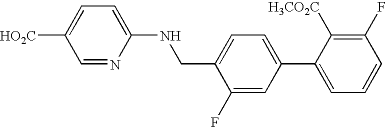

LRMS (ES, M+H⁺): 399

### EXAMPLE 11

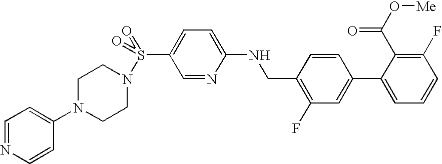

LRMS (ES, M+H⁺): 580

### EXAMPLE 12

### Methyl 3,3′-difluoro-4′-{[(4-{[(2-piperidin-1-ylethyl)amino]sulfonyl}pyridin-2-yl)amino]methyl}-biphenyl-2-carboxylate

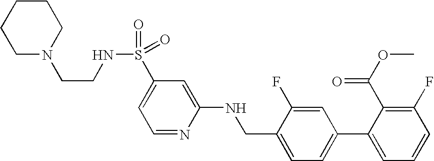

2-Chloro-4-nitropyridine N-oxide (0.99 g, 5.67 mmol), methyl 4′-(aminomethyl)-3,3′-difluorobiphenyl-2-carboxylate (1.05 g, 3.78 mmol), and N-methylmorpholine (1.15 g, 11.35 mmol) were suspended in methanol (7.6 mL) under nitrogen. The reaction vessel was sealed and placed into a 80° C. oil bath for overnight heating and stirring. After 15 hours, the reaction mixture was cooled and concentrated. The residue was subjected to silica gel chromatography eluted with 50% ethyl acetate in hexane to give methyl 3,3′-difluoro-4′-{[(4-nitro-1-oxidopyridin-2-yl)amino]methyl}biphenyl-2-carboxylate.

To a solution of the above material (0.88 g, 2.11 mmol) in methanol (35 mL) was added N-methylmorpholine (0.32 g, 3.17 mmol). Methanethiol gas was bubbled through the solution until saturation. The reaction vessel was sealed and placed in a 100° C. oil bath for 12 hours. The reaction mixture was cooled and concentrated. Silica gel chromatography with 1-10% methanol in methylene chloride provided methyl 3,3′-difluoro-4′-({[4-(methylthio)-1-oxidopyridin-2-yl]amino}methyl)biphenyl-2-carboxylate.

To a solution of the above material (0.72 g, 1.73 mmol) in chloroform (15 mL), at 0° C., was added mCPBA (0.39 g, 2.25 mmol) slowly over 13 minutes. After 50 minutes, calcium hydroxide (0.26 g, 3.46 mmol) was added, and the resulting mixture was stirred for 15 minutes. The mixture was filtered and the filtrate was concentrated to give methyl 3,3′-difluoro-4′-({[4-(methylsulfinyl)-1-oxidopyridin-2-yl]amino}methyl)biphenyl-2-carboxylate.

To a solution of the above material (0.748 g, 1.73 mmol) in methanol (27 mL) was added a 50% aqueous slurry of Raney Nickel (approximately 1 mL). The reaction vessel was purged with nitrogen and then flushed with hydrogen from a balloon. After 2 hours, the reaction mixture was purged with nitrogen prior to filtration through a pad of celite. The filtrate was concentrated to an oily residue, which was subjected to silica gel chromatography eluted with 1-6% methanol in methylene chloride to provide methyl 3,3′-difluoro-4′-({[4-(methylsulfinyl)pyridin-2-yl]amino}methyl)biphenyl-2-carboxylate.

A solution of the above material (0.10 g, 0.24 mmol) in trifluoroacetic anhydride (3 mL) was heated at 50° C. for 3 hours. The mixture was concentrated, and the residue was dissolved in a 1:1 mixture (4 mL) of triethylamine and methanol. After 10 minutes, the solution was concentrated. The residue was again dissolved in methanol and concentrated (×4) to afford methyl 3,3′-difluoro-4′-{[(4-mercaptopyridin-2-yl)amino]methyl}biphenyl-2-carboxylate as an oil, which was used directly in the next reaction without purification.

A solution of the above material (93 mg, 0.24 mmol) in a mixture of ethyl acetate (6 mL) and water (6 mL) was cooled to 0° C. Chlorine gas was bubbled through the solution for 1 minute. The bright yellow solution was partitioned between methylene chloride (80 mL) and water (80 mL), and the organic layer was dried over sodium sulfate, filtered, and concentrated. The oily residue was then dissolved in methylene chloride (4 mL) and cooled to 0° C. 1-(2-Aminoethyl)piperidine (0.19 g, 1.45 mmol) was added, and the resulting mixture was stirred at 0° C. for 1 hour and then warmed to room temperature for overnight stirring. The mixture was subjected to silica gel chromatography eluted with 1-5% methanol in methylene chloride to provide methyl 4′-{[(3,5-dichloro-4-{[(2-piperidin-1-ylethyl)-amino] sulfonyl}pyridin-2-yl)amino]methyl}-3,3′-difluorobiphenyl-2-carboxylate.

A solution of the above material (40 mg, 0.07 mmol) in 2M ammonia in methanol (3 mL) was purged with nitrogen, and 10% Pd/C (16 mg) was added. The reaction vessel was purged with nitrogen and then flushed with hydrogen from a balloon. Additional 10% Pd/C (10 mg) was added every hour for 5 hours until the reaction reached 80% completion. The reaction mixture was purged with nitrogen and then filtered through a pad of celite. The filtrate was concentrated, and the residue was purified by silica gel chromatography eluted with 1-6% methanol in methylene chloride to provide the title compound. HRMS (M+H⁺): calc'd 545.2029, found 545.2047. ¹HNMR (CD₃OD, 400 MHz): δ 8.15 (1H, d, J=5.6 Hz), 7.55 (1H, m), 7.43 (1H, t, J=7.6 Hz), 7.26-7.20 (2H, m), 7.12-7.09 (2H, m), 6.96 (1H, bs), 6.88 (1H, bd, J=5.2 Hz), 4.66 (2H, s), 3.67 (3H, s), 3.03 (2H, t, J=7.2 Hz), 2.40 (2H, t, J=7.2 Hz), 2.36 (4H, bs), 1.54 (4H, m), 1.43 (2H, m)

### EXAMPLE 13

### Methyl 4′-({[4-({3-[4-(4,5-dihydro-1H-imidazol-2-yl)phenoxy]propyl}sulfonyl)pyridin-2-yl]amino}-methyl)-3,3′-difluorobiphenyl-2-carboxylate

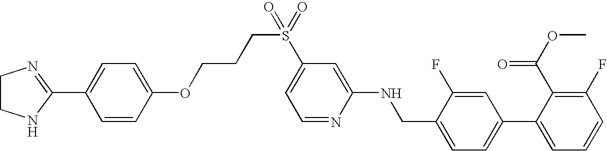

Methyl 3,3′-difluoro-4′-{[(4-nitro-1-oxidopyridin-2-yl)amino]methyl}biphenyl-2-carboxylate (0.500 g, 1.20 mmol, prepared according to Example 2) was dissolved in 3-mercapto-1-propanol (1.2 mL) and methanol (1.2 mL). N-methylmorpholine (0.183 g, 1.81 mmol) was added, and the reaction vessel was sealed and heated at 80° C. overnight. The mixture was concentrated, and the residue was subjected to silica gel chromatography eluted with 4-15% methanol in methylene chloride to yield methyl 3,3′-difluoro-4′-[({4-[(3-hydroxypropyl)thio]-1-oxidopyridin-2-yl}amino)methyl]biphenyl-2-carboxylate.

Into a solution of the above material (0.675 g, 1.47 mmol) in chloroform (15 mL) at 0° was added mCPBA (0.329 g, 1.91 mmol). Additional mCPBA was added periodically during 3 hours to drive the reaction to completion. Calcium hydroxide (0.329 g, 1.91 mmol) was added, and the resulting pink mixture was stirred for 15 minutes prior to filtration. The filtrate was concentrated to provide methyl 3,3′-difluoro-4′-[({4-[(3-hydroxypropyl)sulfinyl]-1-oxidopyridin-2-yl}amino)methyl]biphenyl-2-carboxylate.

A solution of the above material (0.69 g, 1.45 mmol) in methanol (23 mL) was purged with nitrogen prior to the addition of Raney Nickel (approximately 1.5 mL). The mixture was purged with nitrogen and then flushed with hydrogen from a balloon. After 1.5 hours, the mixture was filtered through a pad of celite, and the filtrate was concentrated and dried under vacuum to provide methyl 3,3′-difluoro-4′-[({4-[(3-hydroxypropyl)sulfinyl]pyridin-2-yl}amino)methyl]biphenyl-2-carboxylate.

Into a solution of the above material (0.200, 0.434 mmol) and 4-cyanophenol (0.062 g, 0.52 mmol) in THF (2.2 mL) was added triphenyl phosphine (0.171 g, 0.651 mmol). The resulting mixture was cooled to 0° C., and DEAD (0.113 g, 0.65 mmol) was added dropwise. After stirring 0° C. for 1.5 hours, the mixture was concentrated, and the residue was subjected to silica gel chromatography eluted with 1-3% methanol in methylene chloride to provide methyl 4′-{[(4-{[3-(4-cyanophenoxy)-propyl]sulfinyl}pyridin-2-yl)amino]methyl}-3,3′-difluorobiphenyl-2-carboxylate.

A solution of the above material (0.073 g, 0.13 mmol) in ethanol (4 mL) at 0° C. was saturated with anhydrous HCl. The reaction vessel was sealed and warmed to room temperature for overnight stirring. Solvent was removed, and the residue was dissolved in methanol (2 mL). Ethylene-diamine (0.023 g, 0.39 mmol) was added, and the mixture was stirred at room temperature for 2 hours. Solvent was removed, and the residue was purified by reverse phase Gilson to yield methyl 4′-({[4-({3-[4-(4,5-dihydro-1H-imidazol-2-yl)phenoxy]propyl}thio)pyridin-2-yl]amino}methyl)-3,3′-difluoro-biphenyl-2-carboxylate.

To a solution of the above material (0.022 g, 0.037 mmol) in chloroform (1 mL) at 0° C. was added mCPBA (0.010 g, 0.058 mmol). After 2 hours, the mixture was concentrated, and the residue was subjected to reverse phase chromatography. All fractions with the desired product were combined and lyophilized, and the residue was re-purified by silica gel chromatography eluted with 7-15% methanol (with 10% NH₄OH) in methylene chloride to provide the title compound. HRMS (ES, M+H⁺): calcd 621.1978, measured 621.1991. ¹HNMR(CD₃OD, 400 MHz): 68.22 (bd, J=5.2 Hz, 1H), 7.72 (bd, J=9.2 Hz, 2H), 7.54 (m, 1H), 7.43 (t, J=8 Hz, 1H), 7.23 (m, 2H), 7.10 (m, 2H), 7.02 (bs, 1H), 6.95 (m, 3H), 4.67 (s, 2H), 4.10 (t, J=6 Hz, 2H), 3.79 (s, 4H), 3.66 (s, 3H), 3.43 (m, 2H), 2.17 (m, 2H).

### EXAMPLE 14

### Methyl 3,3′-difluoro-4′-{[(3-{[(2-piperidin-1-ylethyl)amino]carbonyl}phenyl)amino]methyl}biphenyl-2-carboxylate

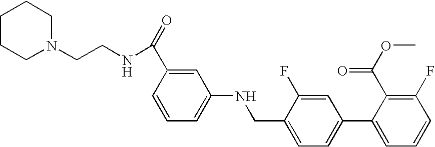

Into a solution of methyl 3-aminobenzoate (0.310 g, 2.05 mmol) in THF (20 mL) was added sodium hydride (0.054 g, 2.26 mmol). The resulting mixture was stirred for 30 minutes, and then methyl 4′-(bromomethyl)-3,3′-difluorobiphenyl-2-carboxylate (0.700 g, 2.05 mmol, prepared according to procedures described in U.S. patent publication no. 2004/0044041) was added. After overnight stirring, additional sodium hydride (0.49 g, 2.05 mmol) was added, and the resulting mixture was heated to reflux for 7 hours. The reaction was quenched by addition of water, and solvent was removed under reduced pressure. The residue was subjected to silica gel chromatography eluted with 15-50% ethyl acetate in hexane to yield methyl 3,3′-difluoro-4′-({[3-(methoxycarbonyl)phenyl]amino}methyl)biphenyl-2-carboxylate.

Into a solution of the above material (0.245 g, 0.60 mmol) in methanol (6 mL) was added 1N NaOH (0.89 mL). After 1 hour, additional 1N NaOH (0.2 mL) was added, and the resulting solution was heated at 35° C. overnight. The pH of the solution was adjusted to pH 6 using 1N HCl, and THF was removed under reduced pressure. The remaining aqueous solution was extracted with two volumes of methylene chloride, and the combined organic solutions were dried over sodium sulfate, filtered, and concentrated to provide 3-({[3,3′-difluoro-2′-(methoxycarbonyl)biphenyl-4-yl]methyl}amino)benzoic acid.

A solution of the above material (0.080 g, 0.20 mmol), 1-(2-aminoethyl)piperidine (0.031 g, 0.24 mmol), 1-hydroxybenzotriazole (0.009 g, 0.06 mmol), and 1-(3-dimethylaminopropyl)-3-ethylcarbodiimide hydrochloride (0.050 g, 0.26 mmol) in methylene chloride (3 mL) was stirred at room temperature overnight. The mixture was subjected to silica gel chromatography eluted with 2.5-7% methanol (with 10% NH₄OH) in methylene chloride. All fractions with the desired product were concentrated, and the residue was re-purified by reverse phase chromatography to provide the title compound as a TFA salt. LRMS (ES, M+H⁺): 508. ¹H NMR (CD₃OD, 400 MHz): δ 7.55 (dt, J=8 and 5.6 Hz, 1H), 7.44 (t, J=7.6 Hz, 1H), 7.23 (m, 3H), 7.10 (m, 4H), 6.85 (m, 1H), 4.48 (s, 2H), 3.73-3.64 (m, 4H), 3.63 (s, 3H), 3.30 (m, 2H), 2.99 (t, J=12.4 Hz, 2H), 1.97 (m, 2H), 1.79 (m, 3H), 1.53 (m, 1H).

The following compounds in Tables 1-3 were prepared by methods analogous to those described in the previous Examples 1-14.

TABLE 1

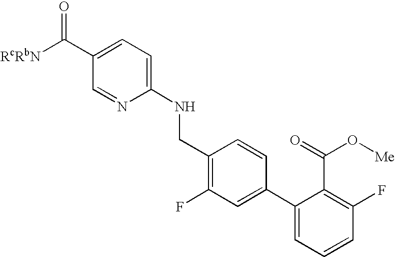

| Example | R^{b} | R^{c} | LRMS (M + H⁺) |
|---|---|---|---|
| 12 | H | 2-(4-morpholinyl)ethyl | 511 |
| 13 | H | 2-(4-pyridyl)ethyl | 503 |
| 14 | H | 4-pyridylmethyl | 489 |
| 15 | H | 2-(N,N-dimethylamino)ethyl | 469 |
| 16 | H | 3-methoxyphenyl | 504 |
| 17 | H | 3-nitrophenyl | 519 |
| 18 | H | phenyl | 474 |
| 19 | Me | phenyl | 488 |

TABLE 2

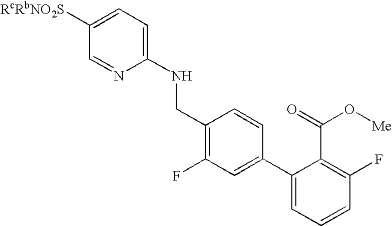

| Example | R^{b} | R^{c} | LRMS (M + H⁺) |
|---|---|---|---|
| 20 | H | 3-methoxyphenyl | 540 |
| 21 | H | 3-methoxycarbonylphenyl | 568 |
| 22 | H | phenyl | 510 |
| 23 | H | 2-methoxyphenyl | 540 |
| 24 | Me | phenyl | 524 |

TABLE 3

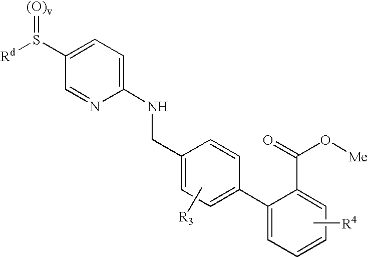

| Example | R^{d} | v | LRMS (M + H⁺) |
|---|---|---|---|
| 25 | 3-methoxycarbonylphenyl | 1 | 537 |
| 26 | 3-methoxycarbonylphenyl | 2 | 553 |
| 27 | Phenyl | 1 | 479 |
| 28 | Phenyl | 2 | 495 |
| 29 | 2-methoxycarbonylphenyl | 1 | 537 |
| 30 | 4-metboxycarbonylphenyl | 1 | 537 |
| 31 | 4-methoxycarbonylphenyl | 2 | 553 |
| 32 | Ethyl | 1 | 431 |
| 33 | Ethyl | 2 | 447 |
| 34 | 2-methoxycarbonylphenyl | 2 | 553 |
| 35 | 4-pyridyl-N-oxide | 2 | 512 |
| 36 | 4-pyridyl | 2 | 496 |
| 37 | 2-methoxyphenyl | 2 | 525 |
| 38 | 2-fluorophenyl | 1 | 497 |
| 39 | 2-methoxyphenyl | 1 | 509 |
| 40 | 2-fluorophenyl | 2 | 513 |
| 41 | 4-carboxyphenyl | 2 | 539 |
| 42 | 2-chlorophenyl | 1 | 513 |
| 43 | 2,4-dichlorophenyl | 1 | 547 |
| 44 | 2-chlorophenyl | 2 | 529 |

### EXAMPLE 45

### 2-({[3,3′-Difluoro-2′-(methoxycarbonyl)biphenyl-4-yl]methyl}amino)isonicotinic acid

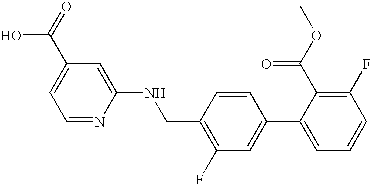

A solution of 2-chloro-4-methyl-5-nitropyridine (5.13 g, 29.73 mmol) in concentrated sulfuric acid (42 mL) was cooled to 0° C., and chromium trioxide (9.81 g, 98.1 mmol) was added. The mixture was stirred at 0° C. for 1 hour and then warmed to room temperature, with an oil bubbler attached, for overnight stirring. The reaction mixture was poured onto ice (300 ml) and diluted with water (150 ml). The mixture was warmed to room temperature, and the solid was filtered and then dried under vacuum to yield 2-chloro-5-nitroisonicotinic acid.

To a stirred solution of the above material (5.3 g, 26.17 mmol) in methanol (50 ml) was added chloroform (200 ml). TMS-diazomethane as a solution in hexane (˜2M) was added dropwise until the color of the reaction mixture remained yellow (˜20 mL). The residual TMS-diazomethane was quenched by addition of acetic acid, and the solvent was removed under reduced pressure. The oily residue was subjected to silica gel chromatography eluted with 50-70% ethyl acetate in hexane to provide methyl 2-chloro-5-nitroisonicotinate.

A solution of the above material (5.66 g, 26.13 mmol), methyl 4′-(aminomethyl)-3,3′-difluorobiphenyl-2-carboxylate (7.971 g, 28.75 mmol, prepared according to procedures described in WO 03/066577), and triethylamine (3.97 g, 39.20 mmol) in methanol (100 ml) was stirred at room temperature overnight. The solution was then heated at 60° C. for 4 hours and cooled to ambient temperature for continued stirring over the weekend. Solvent was removed, and the residue was subjected to silica gel chromatography eluted with 25-50% ethyl acetate in hexane to provide methyl 2-({[3,3′-difluoro-2′-(methoxycarbonyl)biphenyl-4-yl]methyl}amino)-5-nitroisonicotinate as a yellow solid. A solution of the above material (9.3 g, 20.33 mmol) in methanol (330 ml) was purged with nitrogen, and 10% Pd/C catalyst (1 g) was added. The reaction vessel was again purged with nitrogen and then with hydrogen from a balloon. After 23 hours of stirring under hydrogen, nitrogen was bubbled through the solution for 10 minutes prior to filtration through a celite pad. The filtrate was concentrated under reduced pressure to provide methyl 5-amino-2-({[3,3′-difluoro-2′-(methoxycarbonyl)biphenyl-4-yl]methyl}amino)isonicotinate.

Into a solution of the above material (8.45 g, 19.77 mmol) in THF (440 ml) at 0° C. were added hypophosphorous acid (50% solution in water, 110 ml) and sodium nitrite (2.73 g, 39.54 mmol). After 10 minutes of stirring, a catalytic amount of copper (I) oxide was added every 30 minutes for 7.5 hours. The reaction mixture was partitioned between ethyl acetate and water, and the aqueous layer was extracted with additional ethyl acetate. The combined organic layers were washed with saturated sodium bicarbonate and brine, then dried over sodium sulfate, filtered, and concentrated under reduced pressure. The residue was subjected to silica gel chromatography eluted with 20-40% ethyl acetate in hexane to provide methyl 2-({[3,3′-difluoro-2′-(methoxycarbonyl)biphenyl-4-yl]methyl}amino)isonicotinate.

To a stirred solution of the above material (3.96 g, 9.60 mmol) in methanol (85 ml) was added 1N NaOH (11.5 ml), and the solution was heated at 40° C. for 3.5 hours. Solvent was removed under reduced pressure prior to dilution with water. The aqueous solution was washed with diethyl ether twice, and the residual diethyl ether in the aqueous solution was removed under reduced pressure. The aqueous solution was neutralized by addition of 1N HCl (11.5 ml), and the resulting thick suspension was heated (˜70° C.) and then slowly cooled to ambient temperature before being cooled to 0° C. for 30 minutes. The solid was filtered and dried under vacuum, providing the title compound as a white solid. LRMS (ES, M+H⁺): 399. ¹H NMR (CD₃OD, 400 MHz) δ 8.04 (d, J=5.6 Hz, 1H), 7.55 (m, 1H), 7.44 (t, J=8 Hz, 1H), 7.23 (m, 3H), 7.10 (m, 3H), 4.65 (s, 2H), 3.66 (s, 3H).

### EXAMPLE 46

### Methyl 3,3′-difluoro-4′-{[(4-{[(2-piperidin-1-ylethyl)amino]carbonyl}pyridin-2-yl)amino]methyl}-biphenyl-2-carboxylate

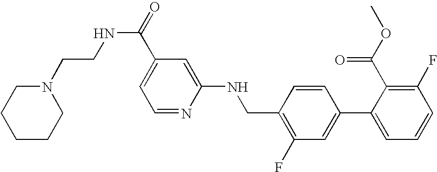

A solution of 2-({[3,3′-difluoro-2′-(methoxycarbonyl)biphenyl-4-yl]methyl}amino)-isonicotinic acid (0.050 g, 0.13 mmol, Example 1), 1-(2-aminoethyl)piperidine (0.032 g, 0.25 mmol), 1-hydroxybenzotriazole (0.019 g, 0.13 mmol), and 1-(3-dimethylaminopropyl)-3-ethylcarbodiimide hydrochloride (0.031 g, 0.16 mmol) in methylene chloride (4 mL) was stirred at room temperature overnight. The mixture was subjected to silica gel chromatography eluted with 1-10% methanol (with 10% NH₄OH) in methylene chloride to provide the title compound. HRMS (ES, M+H⁺): calc'd 509.2359, found 509.2360. ¹HNMR (CD₃OD, 400 MHz) δ 8.05 (1H, bd, J=5.2 Hz), 7.55 (1H, m), 7.43 (1H, t, J=7.6 Hz), 7.23 (2H, m), 7.10 (2H, m), 6.94 (1H, bs), 6.87 (1H, bd, J=5.6 Hz), 4.65 (2H, s), 3.66 (3H, s), 3.52 (2H, t, J=7.2 Hz), 2.57 (2H, t, J=6.8 Hz), 2.51 (4H, bs), 1.63 (4H, m), 1.49 (2H, m).

The following compounds were prepared by methods analogous to those described in the Examples 45-46 and Schemes 1-7.

TABLE 4

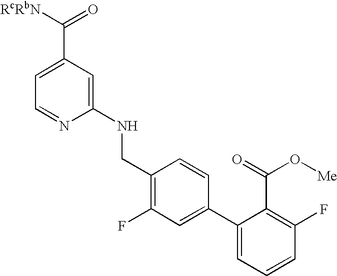

| | | | LRMS |
|---|---|---|---|
| Example | R^{b} | R^{c} | (M + H⁺) |
| 47 | H | 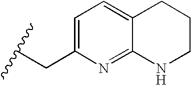 | 544 |
| 48 | H | 3-(4-pyridyl)-1-propyl | 517 |
| 49 | H | 2,3-dihydroxy-1-propyl | 472 |
| 50 | H | 2-methoxycarbonyl-1-propyl | 498 |
| 51 | H | 2-(2-thienyl)ethyl | 508 |
| 52 | H | 2-(4-imidazolyl)ethyl | 492 |
| 53 | H | 2-(4-aminosulfonylphenyl)ethyl | 581 |
| 54 | H | 2-(4-methyl-1-piperazinyl)ethyl | 524 |
| 55 | H | 2-(4-pyrazolyl)ethyl | 492 |
| 56 | H | 2-(2-pyrazinyl)ethyl | 504 |
| 57 | H | 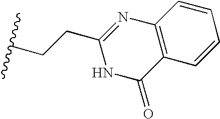 | 570 |
| 58 | H | 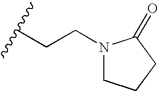 | 509 |
| 59 | H | 2-(5-thiazolyl)ethyl | 495 |
| 60 | H | 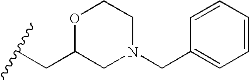 | 587 |
| 61 | H | 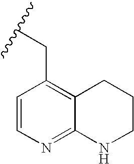 | 544 |
| 62 | H | 1,2,3-triazolylmethyl | 479 |
| 63 | H | 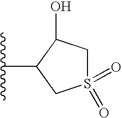 | 532 |
| 64 | H | 3-(1-piperidinyl)-1-propyl | 523 |
| 65 | H | 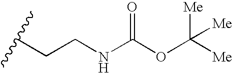 | 541 |
| 66 | H | 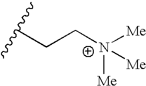 | 484 |
| 67 | H | 2-aminoethyl | 441 |
| 68 | H | 3,3,3-trifluoro-1-propyl | 494 |
| 69 | H | 2-(cyclohexyl)ethyl | 508 |
| 70 | H | l-methyl-2-methoxyethyl | 470 |
| 71 | H | n-propyl | 440 |
| 72 | H | 2-(N,N-diisopropylamino)ethyl | 525 |
| 73 | H | 2-(1,2,4-triazol-1-yl)ethyl | 493 |
| 74 | H | (1-methyl-3-pyrrolidinyl)methyl | 495 |
| 75 | H | 3-tetrahydrofuranylmethyl | 482 |
| 76 | H | 4-tetrahydropyranyl | 482 |
| 77 | H | 2-pyrazinyl | 476 |
| 78 | H | 4-nitrophenyl | 519 |
| 79 | H | 3-nitrophenyl | 519 |
| 80 | H | 4-methoxyphenyl | 504 |
| 81 | H | 3-methoxyphenyl | 504 |
| 82 | H | 4-cyanophenyl | 499 |
| 83 | H | 3-cyanophenyl | 499 |
| 84 | H | 4-(N,N-dimethylamino)phenyl | 517 |
| 85 | H | 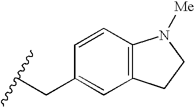 | 543 |
| 86 | H | 4-(trifluoromethyl)benzyl | 556 |
| 87 | H | 3-fluorobenzyl | 506 |
| 88 | H | 3-oxo-3-(t-butoxy)-1-propyl | 526 |
| 89 | H | cyanomethyl | 437 |
| 90 | H | Methyl | 412 |
| 91 | H | 2-carboxyethyl | 470 |
| 92 | Me | 3-chlorophenyl | 522 |
| 93 | H | 3-chlorophenyl | 508 |
| 94 | H | 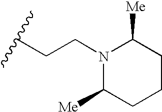 | 537 |
| 95 | H | 2-pyridylmethyl | 489 |
| 96 | H | Phenyl | 474 |
| 97 | H | 2-methoxyphenyl | 504 |
| 98 | H | 3-nitrobenzyl | 533 |
| 99 | H | 3-pyridyl | 475 |
| 100 | H | 4-pyridyl | 475 |
| 101 | H | 3,5-dimethoxyphenyl | 534 |
| 102 | H | 2-pyridyl | 475 |

| | | |
|---|---|---|
| 103 | NR^{b}R^{c}═ | 544 |
| | 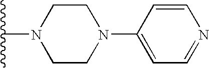 | |
| 104 | 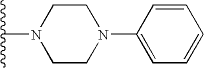 | 543 |
| 105 | 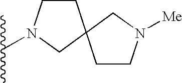 | 521 |
| 106 | 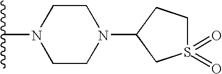 | 585 |
| 107 | 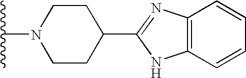 | 582 |
| 108 | 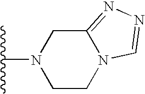 | 505 |
| 109 | 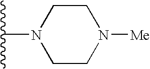 | 481 |
| 110 | 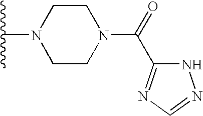 | 582 |
| 111 | 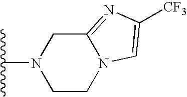 | 572 |
| 112 | 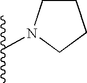 | 452 |
| 113 | 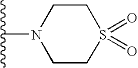 | 516 |
| 114 | 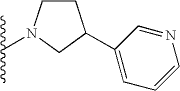 | 529 |
| 115 | 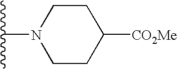 | 524 |
| 116 | 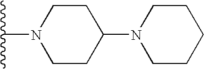 | 549 |
| 117 | 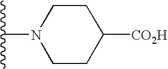 | 510 |
| 118 | 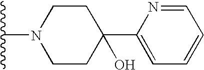 | 559 |

TABLE 5

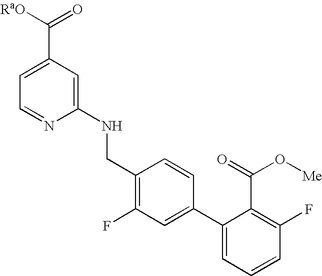

| Example | R^{a} | LRMS (M + H⁺) |
|---|---|---|
| 119 | 2-(1-piperidinyl)ethyl | 510 |
| 120 | 3-methoxyphenyl | 505 |
| 121 | Methyl | 413 |

TABLE 6

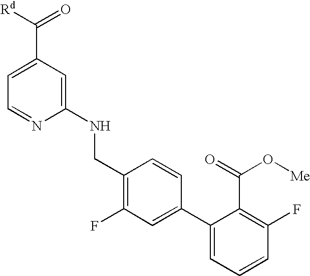

| Example | R^{d} | LRMS (M + H⁺) |
|---|---|---|
| 122 | 5-methy-3-pyrazolyl | 478 |
| 123 | 5-nitro-2-thiazolyl | 526 |
| 124 | 3-methyl-5-isothiazolyl | 495 |
| 125 | 3-pyrazolyl | 464 |

TABLE 7

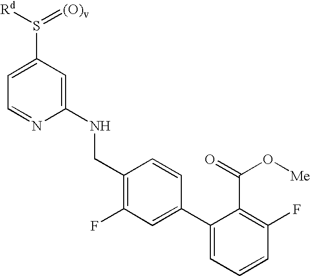

| Example | R^{d} | v | LRMS (M + H⁺) |
|---|---|---|---|
| 126 | Methyl | 1 | 417 |
| 127 | 3-hydroxypropyl | 0 | 445 |
| 128 | 3-hydroxypropyl | 1 | 461 |
| 129 | 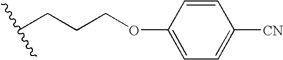 | 1 | 562 |
| 130 | 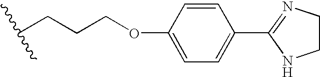 | 0 | 589 |
| 131 | 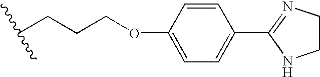 | 1 | 605 |
| 132 | 3-methoxyphenyl | 0 | 493 |
| 133 | 3-methoxyphenyl | 1 | 509 |
| 134 | 3-methoxyphenyl | 2 | 525 |
| 135 | phenyl | 1 | 479 |
| 136 | phenyl | 2 | 495 |

What is claimed is:

1. A compound of Formula I and pharmaceutically acceptable salts thereof: 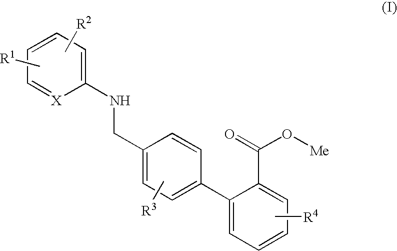
wherein
X is CH or N;
R¹ is selected from (1) NR^{b}SO₂R^{d}, (2) NR^{b}C(O)-phenyl optionally substituted with 1 to 3 groups independently selected from OR^{a}, nitro, halogen, C₁₋₃ haloalkyl, C₁₋₄ alkyl, (CH₂)ₖNR^{b}R^{c}, cyano, SO₂NR^{b}R^{c}, CO₂R^{a}, C(O)NR^{b}R^{c}, 4,5-dihydro-1H-imidazolyl, and C₃₋₆ cycloalkyl, (3) nitro, (4) cyano, (5) 4,5-dihydro-1H-imidazolyl, (6) S(O)ᵥR^{d}, (7) SO₂NR^{b}R^{c}, (8) C(O)Rd, (9) CO₂R^{a}, and (10) C(O)NR^{b}R^{c};
R² is selected from hydrogen, halogen, cyano, nitro, OR^{a}, R^{a}, and C₁₋₄ alkyl, R³ and R⁴ are independently selected from hydrogen, halogen, and C₁₋₄ alkyl optionally substituted with 1 to 5 halogen atoms;
R^{a} is selected from (1) hydrogen, (2) C₁₋₄ alkyl optionally substituted with 1 to 5 groups independently selected from halogen, NR^{e}R^{f} and heterocycle, (3) (CH₂)ₖ-phenyl optionally substituted with 1 to 3 groups independently selected from halogen, cyano, nitro, OH, C₁₋₄ alkyloxy, C₃₋₆ cycloalkyl and C₁₋₄ alkyl optionally substituted with 1 to 5 halogen atoms, (4) C₃₋₆ cycloalkyl, and (5) heteroaryl;
R^{b} and R^{c} are independently selected from
(1) hydrogen,
(2) C₁₋₄ alkyl optionally substituted with 1 to 5 groups independently selected from halogen, heterocycle, cyano, C₃₋₆ cycloalkyl, NR^{e}R^{f}, C₁₋₄ alkyloxy, CO₂R^{a}, OR^{a}, and SO₂R^{d},
(3) (CH₂)ₖ-phenyl, wherein the phenyl is optionally substituted with 1 to 3 groups selected from halogen, cyano, nitro, NR^{e}R^{f}, OR^{a}, CO₂R^{a}, C₁₋₄ alkyloxy, SO₂NR^{e}R^{f}, C₃₋₆ cycloalkyl and C₁₋₄ alkyl optionally substituted with 1 to 5 halogen atoms,
(4) (CH₂)ₖ-heteroaryl,
(5) (CH₂)ₖ—C₃₋₆ cycloalkyl,
(7) (CH₂)ₖ-heterocycle wherein the heterocycle is optionally substituted with one or two groups independently selected from C₁₋₄ alkyl, oxo, benzyl, heterocycle, and OR^{a}, and wherein
the heterocycle is a 4-, 5-, or 6-membered ring containing one or more heteroatoms selected from
NR^{e}, O, and S, wherein the S is optionally oxidized to the sulfone or sulfoxide; or
R^{b} and R^{c} together with the nitrogen atom to which they are attached form a 4-, 5-, or 6-membered ring optionally containing an additional heteroatom selected from NR^{e}, O, and S, wherein the S is optionally oxidized to the sulfone or sulfoxide, and wherein said 4-, 5- or 6-membered ring is (a) optionally fused to benzene or a 5- or 6-membered heteraromatic ring optionally substituted with CF₃, or (b) optionally spirofused to a heterocycle containing N—R^{e}, or (c) optionally substituted with one to two groups selected from heteroaryl, CO₂R^{a}, heterycycle, and OR^{a}; or
R^{b} and R^{c} together with the nitrogen atom to which they are attached form a cyclic imide,
R^{d} is selected from
(1) C₁₋₄ alkyl optionally substituted with 1 to 5 halogen atoms,
(2) C₁₋₄ alkyloxy, optionally substituted with phenyl, wherein the phenyl is optionally substituted with 1 to 3 heterocycle groups,
(3) phenyl optionally substituted with 1 to 3 groups selected from halogen, cyano, nitro, OR^{a}, CO₂R^{a}, C₁₋₄ alkyloxy, C₃₋₆ cycloalkyl and C₁₋₄ alkyl optionally substituted with 1 to 5 halogen atoms,
(4) pyridyl,
(5) heterocycle optionally substituted with one or more heterocycle groups, and
(6) pyridyl N-oxide;
R^{e} and R^{f} are independently selected from hydrogen, C₁₋₄ alkyl, heterocycle, CO₂R^{a}, COR^{a}, phenyl and pyridyl, or
R^{e} and R^{f} together with the nitrogen atom to which they are attached form a 4-, 5-, or 6-membered ring optionally containing an additional heteroatom selected from N, O, and S, wherein the S is optionally oxidized to the sulfone or sulfoxide, and optionally substituted with C₁₋₄alkyl or oxo;
k is 0, 1, 2,3, or 4; and
v is 0, 1, or 2.

2. A compound of claim 1 having the formula I(1) or I(2):

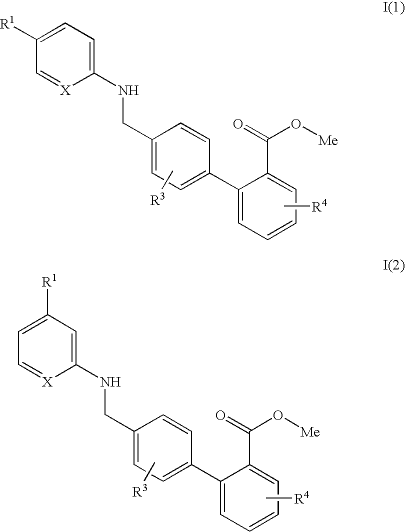

3. A compound of claim 2 wherein X is CH.

4. A compound of claim 2 wherein X is N.

5. A compound of claim 2 wherein each of R³ and R⁴ is halogen.

6. A compound of claim 2 wherein R¹ is C(O)NR^{b}R^{c}, SO₂NR^{b}R^{c} or S(O)ᵥR^{d}.

7. A compound of claim 2 wherein X is N, R¹ is C(O)NHR^{c}, each of R³ and R⁴ is fluorine, and R^{c} is selected from (1) C₁₋₄ alkyl optionally substituted with 1 to 5 groups independently selected from halogen, heterocycle, cyano, C₃₋₆ cycloalkyl, NR^{e}R^{f}, C₁₋₄ alkyloxy, CO₂R^{a}, OR^{a}, and SO₂Rd, (2) (CH₂)ₖ-phenyl, wherein the phenyl is optionally substituted with 1 to 3 groups selected from halogen, cyano, nitro, NR^{e}R^{f}, OR^{a}, CO₂R^{a}, C₁₋₄ alkyloxy, SO₂NR^{e}R^{f}, C₃₋₆ cycloalkyl and C₁₋₄ alkyl optionally substituted with 1 to 5 halogen atoms, and (4) (CH₂)ₖ-heteroaryl.

8. A pharmaceutical composition comprising a therapeutically effective amount of a compound of claim 1 and pharmaceutically acceptable excipients.

9. A method of treatment or prevention of pain and inflammation comprising a step of administering, to a subject in need of such treatment or prevention, an effective amount of a compound according to claim 1 or a pharmaceutically acceptable salt thereof.
